(19) United States
(12) Patent Application Publication
(10) Pub. No.: US 2023/0120693 A1
(43) Pub. Date: Apr. 20, 2023

(54) SGEF CONTROLS MACULAR, CORPUS CALLOSUM AND HIPPOCAMPAL FUNCTION AND DEVELOPMENT, LIVER HOMEOSTASIS, FUNCTIONS OF THE IMMUNE SYSTEM, FEVER RESPONSE ATHEROSCLEROSIS AND TUMOROGENIC CELL GROWTH

(71) Applicant: Pierre Bitoun, Paris (FR)

(72) Inventor: Pierre Bitoun, Paris (FR)

(21) Appl. No.: 17/683,948

(22) Filed: Mar. 1, 2022

Related U.S. Application Data
(63) Continuation of application No. 14/118,817, filed on Nov. 19, 2013, now abandoned.
Application No. 14/118,817 is a continuation of application No. 13/112,788, filed on May 20, 2011, now abandoned.

(51) Int. Cl. A61K 38/17; G01N 33/68; A01N 1/02; C12Q 1/6883

(52) U.S. Cl. CPC A61K 38/1709; G01N 33/68; A01N 1/0226; C12Q 1/6883

(57) ABSTRACT

The invention provides a composition comprising SGEF protein or gene as a therapeutic means to clinical or subclinical defects associated with anomalies of at least one from among the macula, corpus callosum, hippocampus, liver or immune system and diseases including a feverless response to infection, a cancer or vision loss. Methods of diagnosis of such disease and development anomalies are based on detection of mutations of the SGEF gene or altered levels of the SGEF mRNA or protein. A change of at least about 20% in the level of expression visa-vie a normal individual indicates an SGEF anomaly. The SGEF protein is also used as a preventive or curative treatment of atherosclerosis by local or systemic delivery. The invention also provides a composition comprising an inhibitor of the SGEF gene expression or SGEF protein concentration, as a therapeutic means for glaucoma, osteoarthritis, auto-inflammatory diseases, tumors or cancers.

This application is a continuation of U.S. application Ser. No. 14/118,817, filed Nov. 19, 2013, now abandoned, which is a 371 National Stage of PCT International Application No. PCT/US2012/38353, filed May 17, 2012, which is a continuation of U.S. application Ser. No. 13/112,788, filed May 20, 2011, now abandoned.

### SEQUENCE LISTING

The instant application contains a Sequence Listing which has been submitted electronically in ASCII format and is hereby incorporated by reference in its entirety. Said ASCII copy, created on Dec. 7, 2022, is named 078923_559628.txt and is 14,501 bytes in size.

### BACKGROUND OF THE INVENTION

### Field of the Invention

This invention relates to the field of genetics. It identifies a gene, SGEF, which controls the development and function of the retinal macula, the corpus callosum, the hippocampus, the liver, the immune system and inflammation and is a factor in fever response to infections as well as controls cancer and tumor cell formation and metastasis. Null allele mutations in the gene lead to abnormal development and dysfunction, at clinical or sub-clinical levels.

### Description of the Background

Macular retinal dystrophy is a major cause of visual handicap and blindness in children and adults. Several dominant and recessive genetic causes of macular dystrophy have been identified: in vitelliform macular dystrophy or Best disease VMD2 on 1lql13 (1), encoding the bestropin, a chloride channel localized at the basolateral plasma membrane of Retinal Pigment Epithelium (RPE) cells; autosomal recessive Stargardt disease ABCA4 on Ip21-pl3, an ATP binding-cassette transporter whose dysfunction poisons the RPE by accumulation of lipofuscin fluorophores; dominant stargardt-like macular dystrophy ELOVL4 on 6914, encoding a very long chain fatty acid elongase whose dysfunction also causes lipofuscin accumulation in the RPE; North Carolina macular dystrophy localized at 6914-ql6.2, with a variable dominant phenotype with macular drusen and age-related macular degeneration and Bietti's disease with macular crystalline deposits; some rare cases of Stargardt like disease have been associated with mutations in the CNGB3 gene achromatopsia. Nishiguchi, K. M. et al, Hum. Mutat. 25:248-258 (2005). Occult macular dystrophy, a progressive visual disorder, was recently found to be associated with mutations in the RPl-like 1 gene. Akahori, M., et al, Am. J. Hum. Genet. 87:424-429 (2010).

Formation of the human macula is poorly understood. Some of the genes involved in macular development have recently been identified by array CGH. Kozulin, P. et al, Mol Vis 15:45-59 (2009).

Corpus callosum agenesis (CCA) is the most common brain anomaly with a reported incidence of 0.7 to 1 per 1000 live births. CCA has been associated with several gene defects: mutations in L1CAM causing HS AS/MASA syndrome with Hydrocephalus, mental retardation, and adducted thumbs syndrome; in KCC3 causing Andermann syndrome with progressive neuropathy and dementia; in ARX causing XLAG syndrome causing lissencephaly and intractable epilepsy; in MRPS16 causing fatal lactic acidosis with complex I and IV deficiency and brain malformation (Catala M., Neurochirurgie 49(4):441-448 (2003); in ZFHXIB causing Mowat-Wilson syndrome with Hirschsprung disease and cognitive delay; in LRP2 gene causing Donnai-Barrow syndrome with omphalocele, high grade myopia, deafness and nephritis; in WDR2, where gene dysfunction has recently been associated with CCA as well as brain malformations. CCA has also been described as associated with Acrocallosal, Aicardi, Chudley-McCullough, F G, Genito-patellar, Temtamy, Toriello-Carey and Vici syndromes. CCA is occasionally associated with more than 20 other syndromes. About half of these syndromes involve ocular malformations. Paul L K et al, Nat Rev Neurosci. 8(4):287-99(2007).

O'Driscoll recently identified two individuals in one family having 3q25 deletions associated with CCA. O'Driscoll, M. C. et. al, Am. J. Med Genet. A. 152A(9):2145-59 (2010).

The hippocampus and related structures of the medial temporal lobe have a critical role in encoding long-term memory and are also necessary for the maintenance of working memory for novel items and associations including visual memory. Ranganath, C. and D'Esposito M., Neuron. 31(5):865-73 (2001).

Hippocampal hypoplasia has been associated with PROM1, which not only involves macular dystrophy and hippocampus hypoplasia but also cell transformation. Arrigoni F. I. et al, Eur. J. Hum. Genet. 19(2): 131-7 (2011); Zhu L., et. al, Nature, 457(7229):603-7 (2009). Hippocampus hypoplasia and microphthalmia have also been associated with SOX2 mutations. Sisodiya S. M. et. al, Epilepsia 47:534-542 (2006).

Rho proteins are low-molecular-weight GTP-binding proteins which belong to the family of small Rho GTPases and control the cycle between GDP and GTP bound states. Binding of GTP “activates” Rho GTPases by inducing structural shifts that support association of effector molecules that transmit downstream signals. RhoG is an ubiquitously expressed GTPase, which shares significant homology with Rac and binds to a number of the same effector proteins. Gauthier-Rouviere, C. et al, Mol. Biol. Cell 9: 1379-1394 (1998) and Wennerberg K. et al, Biol. Chem. 277:47810-47817 (2002).

SGEF or SH3 (Src Homology 3)-containing Guanine Nucleotide Exchange Factor is a RhoG guanine nucleotide exchange factor that stimulates macropinocytosis (engulfing of extracellular fluid and solute molecules). Ellerbroek, S M. et. al, Mol. Biol. Cell, 15:3309-3319 (2004). Macropinocytosis occurs constitutively in dendritic neural cells for immune surveillance and can be transiently activated in other cells by growth factors. This actin-based process accompanies ruffling of membranes leading to formation of macropinocytic vesicles that engulf large volumes of fluid. This process can be triggered by bacteria (like Salmonella T.), allowing the bacteria to invade cells. Pollard, T. D. and Earnshaw W. C., Cell Biology, Elsevier Science, Saunders Ed. p. 363 (2004).

The Rho GTPase switch. Rho GTPases are targeted to the membrane by posttranslational attachment of prenyl groups by geranyl-geranyltransferases (GGTases). Cycling between the inactive (GDP-bound) and active (GTP-bound) forms is regulated by guanine nucleotide exchange factors (GEFs) which thus accelerate the rate-limiting step of the Rho GTPase cycle and GTPase-activating proteins (GAPs). Guanine-nucleotide dissociation inhibitors (GDTs) inhibit nucleotide dissociation and control cycling of Rho GTPases between membrane and cytosol. Active, GTP-bound GTPases interact with effector molecules to mediate various cellular responses. Upstream activation of the GTPase switch occurs through activation of GEFs. Schmidt A. and Hall, A. Genes Dev. 16: 1587-1609(2002).

It would be desirable to find a single gene/gene product which influences the multiple functions and development of the multiple organs described above. Clearly, that would allow early diagnosis and potential treatment of development or functional problems, as well as diagnosis and potential treatment of development or functional problems that are at the subclinical level.

### SUMMARY OF THE INVENTION

In one aspect of the invention, the invention provides a composition comprising at least one isolated or purified SGEF gene in a functional form and a pharmaceutical carrier introduced into a mammal for expression. The mammal preferably is a human. In one embodiment, the human has a clinical or subclinical condition for at least one disease from among a disease associated with structure or function of macula, corpus callosum, hippocampus, liver, or immune function. The condition is one from among vision impairment, mental impairment, feverless infection, failure to control an infection, or a cancer or tumor state. In another embodiment, the mammal, has a genetic defect causing reduced or null expression or activity of a natural SGEF protein. In one embodiment, the genetic defect causes reduced or null expression or activity of the SGEF gene located at 3q25.2 of the human genome.

In another aspect, the invention provides a composition comprising at least one isolated or purified SGEF protein variant in a functional form. In one embodiment, the variant is a variant of a SGEF protein encoded by a SGEF gene located at 3q25.2. In a preferred embodiment, the variant is at least one SGEF protein variant selected from among the protein variants of SEQ ID No 1, SEQ ID No 2, SEQ ID No 3, SEQ ID No 4, or SEQ ID No 5. More preferably, the at least one SGEF protein variant is selected from among the protein variants of SEQ ID No 1, SEQ ID No 2, or SEQ ID No 3. In accordance to one embodiment, the SGEF protein or variant thereof is introduced as a genetic construct for expression in the mammal.

In yet another aspect, the invention provides a method of treatment comprising providing at least one SGEF protein variant and a pharmaceutical carrier to an individual manifesting a clinical or subclinical condition or predisposition for a disease associated with functional or structural defects corresponding to retina/macula anomaly (“RMA”), corpus callosum anomaly, hippocampus anomaly, liver disease, immune response deficiency or feverless infection. Preferably, the SGEF protein is a SGEF protein corresponding to the protein encoded by the SGEF gene located at 3q25.2. In accordance to one embodiment, the disease state is associated with RMA and comprises at least one disorder from among retinal disorders, macular disorders, macular dystrophies or macular degenerations like age-related macular degeneration, geographic atrophy, diabetic retinopathy, glaucomatous retinal dysfunction or disease of any part of the eye and visual disorders. In accordance to another embodiment, the disease state is associated with corpus callosum anomaly and comprises at least one disorder from among hypoplasia, absence or thickened corpus callosum and coordination disorders, including hand eye coordination disorders. In yet another embodiment, the disease state is associated with hippocampal development deficiency or dysfunction and comprises at least one disorder from among memory dysfunction, intellectual deficiency, mental retardation, Alzheimer disease or degenerative brain disorders. In a further embodiment, the disease state is associated with immune response and comprises at least one disorder from among innate or acquired immune deficiency disorder caused by HIV infection, congenital immune deficiencies, ADA (adenosine deaminase), or steroid induced immune deficiency. In a further yet embodiment, the disease state is associated with liver disease and comprises at least one disease from among hepatitis, congenital liver disease, liver cirrhosis or lack of liver homeostasis.

In yet another aspect, the invention provides a method of diagnosis of at least one disease state selected from among retinal macular anomaly (RMA) or any part of the eye, corpus callosum anomaly, hippocampus anomaly, liver disease, immune dysfunction, or feverless response to infection, comprising identifying a defect in an SGEF gene located at 3q25.2 or reduction in the concentration of an SGEF protein. Preferably, the identification is by hybridization to a probe specific for the SGEF gene, PCR analysis or quantitative PCR (qPCR) and western blot analysis. In accordance to one embodiment, the diagnosis of an individual comprises the detection of a defect in the SGEF gene located at 3q25.2 in a consanguineous other individual or manifesting clinical or physical anomaly corresponding to at least one disease state from among retinal macular anomaly (RMA) or any part of the eye, corpus callosum anomaly (CCA) liver disease, immune dysfunction and feverless response to an infection. In accordance to another embodiment, the defect causes a change of at least about 20% in the level of expression of SGEF RNA or protein.

In a yet still another aspect, the invention provides a method of treatment or prevention of atherosclerosis or arteritis of all arteries and more specifically coronary artery disease comprising the modulation of SGEF expression or activity by genetic or pharmacologic means in a mammal.

In a further aspect, the invention provides a method of treatment or prevention of cancer or tumor growth, comprising administration of an agent to reduce SGEF presence or activity in a mammal. In one embodiment, the invention provides a method for prevention of cancer or tumor growth, wherein the cancer or tumor is a prostrate, brain, breast, ovary, oesophageal, gastrointestinal, liver or yet other cancer or tumor.

In still another aspect, a method of treatment is provided, wherein an SGEF inhibitor is provided to a subject. In one example, the invention provides a method of treatment or prevention of osteoarthritis and joint inflammatory processes, comprising administration of an agent to reduce SGEF presence in a mammal, the agent being administered locally or systemically. In another example, the invention provides a method of treatment or prevention of inflammatory or auto-inflammatory diseases, illnesses or processes, comprising administration of an agent to reduce SGEF presence or activity in a mammal, the agent being administered locally or systemically. In yet another example, the invention provides a method of treatment or prevention of a cancer state or tumor growth. For example the cancer or tumor may be a prostate, a brain, a breast, an ovary or a liver cancer or tumor.

In a further aspect, the invention provides a method of treatment or prevention of increased intraocular pressure or glaucoma comprising administration of an agent to reduce SGEF presence in a mammal the agent being administered locally and or systemically.

In a yet further still aspect, the invention provides a method of preservation or preparation of an organ for transplantation, wherein said organ is exposed to a solution comprising SGEF protein or protein variant. In accordance to one embodiment, the organ is liver.

In a still yet further aspect, the invention provides a kit for treatment of a patient comprising at least a functional domain of an SGEF protein and a pharmaceutical excipient. In accordance to one embodiment, the protein is provided as a gene for expression in a mammal.

In a yet still further aspect, the invention provides a kit for treatment of a patient comprising at least an inhibitor of an SGEF protein functional domain and a pharmaceutical excipient.

### DETAILED DESCRIPTION

### Drawings

FIG. 1 depicts a genetic analysis identifying the locus of the deletion mutation. The upper panel presents a Comparative Genome Hybridization (CGH) analysis. The probes are indicated as dots. This figure shows the line shift on chromosome 3q25.2 indicating the homozygously deleted genetic material indicated by the missing hybridized probes in this region.

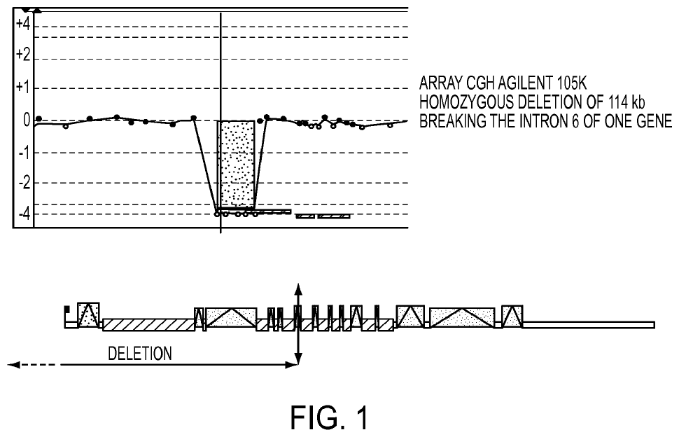

The lower panel of FIG. 1 is a depiction of the SGEF genetic locus. The single line illustrates introns. The boxes represent exons. The black arrow at bottom shows the missing upstream region and the first six exons which are removed by the deletion.

### DESCRIPTION

Individuals carrying a homozygous null allele SGEF gene mutation have been identified. Unexpectedly, the mutation has revealed the role of SGEF in development of organs and control of multiple functions. The invention provides therapeutic and diagnostic options based on the SGEF gene and protein. The gene mutation reveals the SGEF gene and its product to affect structures and/or functions shown to be associated with retinal macular development; corpus callosum development; hippocampal development; liver function, immune function, and lack of fever response. In contrast, the excess of expression of the gene or increased protein level increases certain cell multiplication and cell transformation, to play a role in tumor or cancerous growths and atherosclerosis. Under-expression of SGEF controls the development of other tumor or cancerous growths.

This is an unexpected development, because patients simultaneously impaired in these functions are rarely observed. Furthermore, cases of one genetic locus affecting these multiple structure and functions are not known. Without limiting the invention to any theory as to a mechanism of action, it is noted that SGEF is an activator of RhoG, a GTPase protein. There are many known GTPases and the absence of one GTPase regulator might have been considered insufficient to cause multiple syndromes, because it might have been expected that there are separate control mechanisms for GTPase associates with particular functions and/or tissues and, furthermore, it would have been expected that there is redundancy in the control of individual classes of GTPases. There are more than 60 known human Rho GEFs (out of 85 GEF's in the human genome) and each particular GEF determines in which membrane the GTPase is activated and, by acting as a scaffold, which downstream protein the GTPase activates. Alberts B. et al, Molecular Biology of the Cell, Garland Science, N. Y, 5th edition, 927, 931, 1043 (2008). Likewise, and again without limiting the invention to any mechanism of action, it is noted that SGEF and ICAM1 are also working in tandem, are part of a common genetic pathway responsible for multiple phenomena, van Buul J D, et al “RhoG regulates endothelial apical cup assembly downstream from ICAM1 engagement and is involved in leukocyte trans-endothelial migration” J. Cell Biol. 178(7): 1279-93 (2007). Further yet, in certain genetic pathways, SGEF is expected to coordinate functions with both the ICAM1 and the RhoG genes.

Nonetheless, a mutation was now shown to have multiple effects. A combination of genetic analysis and clinical and physical observations confirmed the role of the gene. The gene defect was shown to be responsible for defects in both homozygous and heterozygous individuals, establishing its overall role in control of development and function of multiple systems. This points to SGEF dosage sensitivity in different parts of the cell at different times and in different tissues to allow proper development and function of the retinal macula, the corpus callosum, the hippocampus, the liver and immune function, and normal fever response to infection.

Relying on array Comparative Genetic Hybridization (“CGH”) analysis (e.g. as available from Agilent Technologies), the inventor determined that consanguineous parents (cousins) are each heterozygous for a deletion mutation in the SGEF gene located at 3925.2. The results were confirmed by quantitative PCR analysis. The parents produced three children and all the children are homozygous for the deletion mutation at the 3q25.2 locus. The inventor determined the deletion to cover the same region in all the family members. It is an about 114 kilobase deletion, comprising the 5′-end of the gene including the promoter region and extending into the 6^{th} intron of the SGEF gene and thus abolishing the gene function linked to this major promoter.

The oldest child of this family is Child 1, the middle child and the proband for the genetic study is Child 2. No gross genetic defects were observed upon peripheral lymphocyte karyotypic analysis of the family members.

The proband (Child 2) was initially analyzed for genetic defects because of severe symptoms of retinal dystrophy, macular degeneration and macular dystrophy, resembling a severe and congenital form of Stargardt's disease. Accordingly, this child and the family members also were analyzed for genetic defects at the ABCA4 (previously called ABCR) locus, a locus known to be associated with Stargardt's disease. Allikmets R., Nat. Genet. 17(1): 122 (1997).

To help rule out the coincidence of the mutation at 3q25.2 existing in the background of known mutations associated with macular dysfunction or abnormalities, the proband was tested also for mutations in 18 other known autosomal recessive Retinitis Pigmentosa genes: CERKL, CNGA1, CNGB1, MERTK, PDE6A, PDE6B, PNR, RDH12, RGR, RLBP1, SAG, TULP1, CRB, RPE65, USH2A, USH3A, LRAT, and PROML1. The proband was shown to have two variant isoforms in the ABCA4 locus on the same chromosome (IVS45+7G>A and S2255I) paternally inherited, but no mutations in the other 18 loci. Accordingly, the other four family members were tested for the ABC4 locus mutation. Only the father had the two ABCA4 variants, which he transmitted to the proband. While the S2255I variant is likely a polymorphism, the role of the splice site variant is debated. Valverde, D. et al, Invest Ophthalmol. Vis. Sci. 48(3):985-90 (2007). ELOVL4 was also sequenced in proband and without any detectable mutation.

Clinical observation and/or testing revealed the following phenotypes and morphologies. The proband had congenital nystagmus and vision impairment with congenital macular dystrophy shown upon fundus examination. Optical Coherence Tomography imaging showed reduced thickness of the retinal macula (92μη, i.e. about 50% of normal). Further evidence of macular dysfunction and dystrophy were documented by Visual Evoked Potential (VEP) analysis which record visual occipital cortex activity (using occipital cranial electrodes) elicited by light stimulation of each eye.

Prenatal ultrasound had also demonstrated Corpus Callosum (“CC”) agenesis. Magnetic Resonance Imaging demonstrated the complete absence of axonal corpus callosum fibers (white matter) and diminished volume of hippocampus gray matter (hypoplasia), as well as external hydrocephalus (excessive fluid volume outside the brain) were observed in the proband. The proband demonstrated reading and learning difficulties, conditions expected in view of these physical defects.

The proband also had protracted EBV infection lasting for months and the associated mononucleosis, causing severe liver damage (hepatic cytolysis), as well as simultaneous Group A beta-hemolytic streptococcus infection without ever showing signs of fever. The observation regarding the infection and the lack of fever conceptually fits the known function of SGEF in dorsal ruffles formation, i.e. suggesting a trans-endothelial migration role that is involved in the immune response. Accordingly, the immune response is affected by the 3925.2 locus mutation (SGEF gene). The proband has evidenced the lack of ability to mount a fever response to multiple serious infections including at least a protracted, three-months course of infectious mononucleosis complicated by liver involvement, Group A beta streptococcal infection, tooth abscess, upper respiratory infection etc. This lack of fever is clearly linked to the immune role of SGEF.

Because of the high level of brain expression of SGEF and its role in the cortical and white matter it is likely that SGEF dysfunction mediates the lack of fever which would normally be an aspect of the multiple serious infections observed in the proband. Without limiting the invention to any particular mechanism of action, it is noted that recurrent fevers have been associated with several disorders involving inflammatory conditions, including Familial Mediterranean fever linked to the MEFV gene. Cell 90:797-807 (1997); Houten, S. M. et al, Nature Genet. 22:175-177, (1999). Dominant periodic fever has been associated with the Tumor Necrosis Factor Receptor Super Family 1A, TNFRSF1A. McDermott, M. F. et al, Cell 97:133-144 (1999). A spectrum of auto-inflammatory conditions, the cryopyrinopathies, have been linked to mutations in Cryopyrin, the protein encoded by CIAS 1, which activates Caspase 1, which in turn causes release of the active pro-inflammatory cytokine interleukin-lbeta. IL-lbeta Ryan J. G. and Kastner, D. L., Curr. Topics Microbiol. Immunol. 321:169-84 (2008).

Lack of fever has been previously observed in familial dysautonomia also known as Riley-Day syndrome which is, like SGEF, involved with cytoskeletal regulation. Cheishvili D. et al, Hum. Mol. Genet. 2011 Feb. 11. [Epub ahead of print.]

Basal body temperature has been linked to serotoninergic receptors 5-HT (1 A). Olivier J. D. et al., Eur. J. Pharmacol. 20:590(1-3): 190-7 (2008). Basal body temperature is mediated by an 5-HT(1 A) receptor population. Bacterial and viral infections induce Hypothalamic Pituitary Axis activation, and also increase brain Nor Epinephrine and 5-HT metabolism and brain tryptophan. These effects are strikingly similar to those of IL-1, suggesting that IL-1 secretion, which accompanies many infections, may mediate the Nor Epinephrine and 5-HT metabolism and brain tryptophan responses, possibly via the Serotoninergic receptor and IL1 activation. Dunn A. J., Clin. Neurosci. Res. 6(1-2):52-68 (2006).

Accordingly, the SGEF protein has multiple pathways available to affect fever, any one of them likely involving an effect on a cellular receptor site or a second messenger agent or possibly the control of leukocyte transendothelial migration.

Furthermore, the defective immune response in part explains the severity of the liver damage. Furthermore, however, the SGEF protein also has a role in liver homeostasis. Dysregulation is an effect of the null allele SGEF mutation. SGEF is highly expressed in the liver (more than in other tissues). Ellerbroek, S. M. et al, Mol Biol. Cell 15:3309-3319 (2004). Therefore, the unusually extensive damage of the liver upon EBV infection points out to a role for SGEF in liver homeostasis. (No limitations of the invention in respect to the mechanism of action are implied by these observations by the inventor.)

Child 1 was shown to carry the same SGEF gene homozygous deletion as Child 2 but has no defects in the ABC4 gene. Albeit his vision and OCT tests were normal, multifocal electroretinogram (ERG) (which records retinal electrical activity of the central part of the retina using corneal, frontal and temporal electrodes during light stimulation of each eye) (focused on fovea, the center of the visual axis) showed a severely dysmorphic poorly developed fovea bilaterally. No liver study was performed on Child 1.

Accordingly, albeit Child 2 had two isoform variants in the ABC4 locus, the macula development defect was at least in part caused by the SGEF defect, as Child 1 had the macular structural defect but no ABCA4 mutations. Furthermore, the fovea and CC abnormality were seen in both of the two children having a common homozygous gene condition defect.

Child 3 was subsequently found to present with low vision of 20/200 bilaterally with the same macular dystrophy visible on fundus examination at the age of four years. OCT examination revealed similar absence of foveal pit, thinning of the retina with interruption of photoreceptor layer and poorly developed macula. She did not have any brain anomaly on MRI and demonstrated no nystagmus.

The father is heterozygous for the 3q25.2 deletion and had the two ABCA4 locus variant isoforms. The MRI results were normal for corpus callosum, and the hippocampus. Multifocal ERG revealed the fovea of one eye was affected, with significantly reduced foveal cone function. The observations that SGEF defects lead to deficiency in foveal cone function is consistent with a conclusion that SGEF is responsible for neuronal and possibly blood vessel guidance—when SGEF protein is absent, the neuron and/or blood vessel deviate in their growth path, invade the fovea and interfere with cone formation and/or function. The effect is seen even in a heterozygous individual for the gene defect. Cell surface receptors like the ephrin receptor tyrosine kinase at the surface of neurons have been shown to activate the GTPase RhoA via the Rho GEF ephexin to cause myosin-dependent contraction of the actin filament cytoskeleton and to thus cause growth cone collapse of the axon tip. Alberts B. et al, supra, at page 921-22. The father was also shown by erg multifocal analysis to have defective foveal cone function unilaterally. (No limitation of the invention with respect to the mechanism of action is implied by these observations by the inventor.)

The mother, who is heterozygous for the 3q25.2 locus deletion was not shown to harbor defects in the hippocampal or CC development, but had granular ocular fundi. Again, a heterozygous individual was nonetheless at least partially affected. The precise boundaries of the SGEF deletion observed were exactly identical both in the heterozygous parents and the three homozygous offspring this eliminating any boundary effect. The chromosomal deletion was within a 32 megabase region of homozygosity.

These cumulative observations on this family are summarized in Table 1. (In Table 1, ND stands for no defect found. NT stands for not tested.) The top three rows of Table 1 summarize the results of genetic analysis results for the loci in the left-hand column. The reminder of Table 1 refers to the clinical or physical exam observations in the respective patient, as related to the defect or the tissue indicated in the left-hand column.

Accordingly, although there are differences in the severity of the conditions, the SGEF has a mediating role in proper development of the macula and in particular the fovea, the CC, the hippocampal region and immune response and liver homeostasis, a role observed in both homozygous and heterozygous individuals. Absence or reduced amount of the SGEF gene product produces the medical effect enumerated here.

TABLE 1

| | | | | Child 2 - | |
|---|---|---|---|---|---|
| | Mother | Father | Child 1 | proband | Child 3 |
| 3q25.2 locus | Hetero- | Hetero- | Homo- | Homo- | Homo- |
| deletion | zygous | zygous | zygous | zygous | zygous |
| ABCA4 locus | ND | Two variant | ND | Same two | ND |
| variant isoforms | | isoforms | | isoforms as | |
| | | present. | | in father are | |
| | | | | present. | |
| RP genes, 18 | NT | NT | NT | No defects | NT |
| loci | | | | observed. | |
| Vision, macula, | Granular | Fovea of | Fovea of | Multiple | Multiple |
| fovea, | texture | one eye is | both eyes | functional | functional |
| development | of fundi | defective | defective | defects and | defects and |
| and function | | on Mf ERG. | on Mf ERG. | fovea and | fovea and |
| defects. | | | | fundus | fundus |
| | | | | structural | structural |
| | | | | defects. | defects. |
| CC | ND | ND | MRI | Complete | ND |
| development, | | | revealed | absence, | |
| function and | | | small | reduced axon | |
| structure. | | | defect. | white matter | |
| Hippocampal | ND | ND | ND | Hypoplasia, | ND |
| development | | | | reduced gray | |
| and function | | | | matter | |
| Immune | NT | NT | NT | Streptococcus A | Multiple |
| function and | | | | and EBV | dental |
| fever response | | | | infections; | abcesses; |
| defects. | | | | dental abcesses; | lack of |
| | | | | lack of fever. | fever. |
| Liver | NT | NT | NT | Severe liver | NT |
| homeostasis. | | | | damage. | |

A series of patients were tested for the 3q25.2 deletion including the family reported by Descartes et al., supra, who described a brother and sister with non-documented Stargardt's disease and CCA (but with more severe handicap involving facial dysmorphism, mental retardation and deafness). A second family with retinal dystrophy, CCA and mental retardation was also tested. Using DNA sequencing and quantitative PCR, both families were shown to be negative for SGEF involvement. A 100 patient cohort affected with Aicardi syndrome and other CCA patients, various macular dystrophy phenotypes, ABCA4 mutation-negative Stargardt disease patients as well as Age-related Macular Dystrophy (AMD) patients were genotyped using sequencing, but no SGEF mutations were identified. Therefore, the 3q25.2 SGEF gene is not the only gene locus responsible for syndromes affecting the retinal macular, corpus callosum and hippocampal development and immune function. Nonetheless, insufficient SGEF also has a negative role in the development of these systems and functions.

The macular foveal development is conditioned by the lack of blood vessel entry and highly dense cone photoreceptor enrichment, critical to the spatial resolving power of the fovea, where cone inner segment spacing reaches a peak of 100,000 to 300,000 mm⁻². Curcio C. A. et al., J. Comp. Neurol. 292:497-523 (1990).

Without limiting the invention to a particular mechanism of action, it should be noted that a method of interaction between SGEF and Phosphoinositide 3-kinase (PI3K) is apparent. In particular, P13K constitutes docking sites for the plekstrin homology (PH) domain of SGEF. PI3K is a lipid kinase that phosphorylates phosphatidyl inositides in lipid bilayer membranes. The role of the SGEF deletion in causing macular cone dysfunction is therefore supported by the recent finding that cone dystrophy has been described in association with PI3K deficiency in mice PI3K is a classic survival kinase linking extracellular trophic/growth factors with intracellular anti-apoptotic pathways Ivanovic, I. et al, Invest. Ophthalmol. Vis. Sci., 2011, March [Epub ahead of print] PMID:21398281.

Provis, J. M. et al, Association for Research in Vision and Ophthalmology annual meeting, poster 4014/A125 (2009) discussed whether the critical lack of development of blood vessels in the macula is due to the role of axon guidance genes controlled by an interaction with netrin-UNC5 or Ephrin-6 repelling their growth in the macula and particularly into the fovea or only due to anti-angiogenic factors. Ephrin 6A seems to be present in the ganglion cell layer of fetal macaque retina in incremental axial Posterior to anterior gradient concentration to the fovea thus repressing entry of endothelial cells and blood vessels into the foveal region of the retina according to Provis et al, Id. The presence of Ephrin6A (a neuronal guidance gene) gradient would thus be a factor that blocks blood vessel entry into the retina as could be discussed with regards to SGEF (which is clearly also playing a role in neuronal guidance as evidenced by the fact that its deficiency causes ACC).

The situation where a congenital macular anomaly is a sign of a developmental defect presenting as an early onset macular dystrophy is linked to complete lack of function of SGEF in the fetal retina, which thus indicates a role for this gene in embryonic and early post-natal macular development as we know that macular development continues postnatally.

The association of corpus callosum agenesis in the homozygous null allele is consistent with a role for SGEF in axon guidance at the level of the interhemispheric fissure interacting with the L1CAM gene product cited above and possibly the HESX1 gene product (which controls the septum pellucidum (a white matter midline brain structure formation) or BMP Bone Morphogenic Protein signaling which have all been shown to mediate Corpus callosum formation. Paul, supra. The external hydrocephalus observed in the proband associated with the SGEF homozygous null allele is more evidence pertaining to the role in axon guidance in meningeal development because the outer brain meninges are the site of the external brain fluid control.

During fetal brain development, axons growing from pyramidal neurons of cortical layer III extend and cross the midline. In experimental models, e.g. mice, it is possible to decipher two conditions in which the development of the corpus callosum is impaired. The first condition is characterized by an impairment of the formation of the roof of the telencephalon (the primordium of the commissural plate). This condition can be explained by an abortive induction of this region by an impairment of BMP signaling. This can generate all the forms of holoprosencephaly. Other forms are due to a defective gene encoding hesx1, a transcription factor involved in the control of telencephalic morphogenesis. Such a genetic defect in HESX1 can be observed in human dominant forms of septo-optic dysplasia. The second condition is explained by an impairment of the molecular control of axon growth: such is the case for the couple netrin1 and DCC or for the adhesion molecule L1cam.

Other genes originally identified by their involvement in axon patterning are also implicated in vascular patterning. The semaphorin-plexin family of genes shares with VEGFA the capacity to bind neuropilin1, expressed by both blood vessels and axons. Class 3 semaphorins are also known to have a repellent effect during vascular morphogenesis via interactions with integrins. Serini, G. et al, Nature 424:391-7 (2003). Eph receptors and their ephrin ligands have key roles in axon guidance, provide guidance cues for endothelial cells during development, are involved in assembly and maintenance of vascular networks, and arteriovenous differentiation. Pfaff, D. et al, J. Leukoc. Biol. 80:719-26 (2006). Netrin is a potent vascular mitogen and has a role in repelling developing vessels via interactions with the UNC5 receptor, while Slit2 is implicated in endothelial cell migration Park, K. W. et al, Proc. Natl. Acad. Sci. USA 101: 16210-5(2004); Suchting, S. et al, Exp. Cell Res. 312:668-75 (2006). Of particular interest are the repellent effects of netrin-UNC5 interactions and Eph-ephrin signaling on developing vessels as well as axons during development Lu, X. et al, Nature 432: 179-86(2004); Cowan C. A. et al, Trends Cell Biol. 12:339-346 (2002). Accordingly, we conclude that a graded expression of genes involved in repellent signaling that is centered on the fovea during development—similar to the one reported for Eph-A6—retards the growth of vessels into the central region of the retina, and contributes to definition and developmental pattern of the foveal avascular area.

Bacterial pathogens such as Salmonella Typhimurium use RhoG activation to enter the host cell. Patel J. C. and Galan, J. E., J. Cell Biol. 175(3:453-63, Epub (2006). The authors performed an RNA interference screen for Rho GTPases that could account for SopB-dependent invasion. They found that knockdown of RhoG resulted in reduced levels of serovar Typhimurium invasion. RhoG was activated and recruited to sites of serovar Typhimurium invasion in a SopB-dependent manner. Next, they investigated how SopB activates RhoG and discovered that SGEF (SH3-containing guanine nucleotide exchange factor) was recruited to ruffles in a SopB-dependent manner and that it was required for SopB-dependent RhoG activation. These observations are consistent with the role of SGEF in preventive bacterial infection and the mutant gene being defective in bacterial rejection, as noted in the present invention. The defect can be remedied by provision of SGEF gene or gene product.

The SGEF gene/protein are also known by other names, i.e. cSGEF for a terminal 3′ isoform; HMFN1864; DKFZp434D146; and ARHGEF26. See, www.ncbi.nlm.nih.gov/pubmed?db=gene&Cmd=retrieve&dopt=full_report&list_u ids=26084, last viewed on Jan. 31, 2011. Diversification of transcriptional modulation: large-scale identification and characterization of putative alternative promoters of human genes, Kimura, K. et al, Genome Res. 16(1):55-65 (2006); Expression profiling and differential screening between hepatoblastomas and the corresponding normal livers: identification of high expression of the PLK1 oncogene as a poor-prognostic indicator of hepatoblastomas, Yamada, S. et. al., Oncogene, 23(35):5901-11 (2004); SGEF, a RhoG guanine nucleotide exchange factor that stimulates macropinocytosis, Ellerbroek S M, et. al., Mol. Biol. Cell, 15(7):3309-19 (2004); Ota T. et. al; Nat Genet. 36(1):40-5 (2004); Isolation of the novel human guanine nucleotide exchange factor Src homology 3 domain-containing guanine nucleotide exchange factor (SGEF) and of C-terminal SGEF, an N-terminally truncated form of SGEF, the expression of which is regulated by androgen in prostate cancer cells, Qi H. et al., Endocrinology, 144(5): 1742-52(2003), and Generation and initial analysis of more than 15,000 full-length human and mouse cDNA sequences, Strausberg R. L. et al, Proc. Natl. Acad. Sci. U.S.A. 99(26):6899-903 (2002).

A summary of the gene's information is found in Thierry-Mieg, Danielle and Thierry-Mieg, Jean, Genome Biology 7(1):S12 (2006), or online at www.ncbi.nlm.nih.gov/IEB/Research/Acembly/av.cgi?db=human&l=SGEF, last viewed on Feb. 10, 2011. The complete gene sequence is available on Genebank under accession number AC_000046.1, using the GRCh37.p2 primary reference assembly: www.ncbi.nlm.nih.gOv/nuccore/NC_000003.11?from=153839149&to=153975616 &report=genbank, last viewed on Mar. 7, 2011.

The gene is ubiquitously expressed and is expressed at higher than average levels. It has particularly high expression in retina, brain and liver. As noted above, the gene sequence information as well as the locations of exons and introns are known. The sequences of mRNAs isolated from various tissues are also known. Deduced amino acid products are provided. Alternative SGEF protein variants are produced, depending on alternative expression and processing, e.g. splicing and choice of transcriptional promoter. There is sufficient information to allow an artisan skilled in the art using known methods to construct an artificial gene and vector for expression of an SGEF protein and variants.

In accordance to one aspect of the invention, an SGEF protein is provided to a mammalian patient. Preferably, more than one SGEF protein is provided to a mammal. Alternatively, an artificial SGEF gene may be expressed in a mammal or the genes encoding variants may be co-expressed in the mammal. In another alternative, homologs or isoforms of SGEF proteins or genes are also within the scope of the invention. (The expression “is/are within the scope of the invention” herein means that the construct or step discussed provides the construct or step required to achieve the goal of the invention.) For example, an SGEF construct within the scope of the invention is a construct that supplies SGEF in SGEF deficient recipient mammal or to increase the overall expression of SGEF in a recipient which already expresses a form of SGEF.) Homologs of SGEF are SGEF proteins from species other than Homo sapiens. Any gene or protein constructs (corresponding to a sequence larger than about 90 amino acids, up to about 900 amino acids) based on the SGEF gene sequence or from the prototype sequences listed below, are within the scope of this invention.

According to a preferred embodiment, as an example the artificial SGEF gene encodes a protein which is 751 amino acids (“aa”) in length. The preferred prototype 751 a.a. protein sequences is:

(SEQ ID NO: 1)
MDGESEVDFSSNSITPLWRRRSIPQPHQVLGRSKPRPQSYQSPNGLLITD
FPVEDGGTLLAAQIPAQVPTASDSRTVHRSPLLLGAQRRAVANGGTASPE
YRAASPRLRRPKSPKLPKAVPGGSPKSPANGAVTLPAPPPPPVLRPPRTP
N APAPCTPEEDLTGLT ASP VPS PT ANGL A ANNDS PGS GS QS GRKAKDPER
GLFPGPQKSSSEQKLPLQRLPSQENELLENPSVVLSTNSPAALKVGKQQI
IPKSLASEIKISKSNNQNVEPHKRLLKVRSMVEGLGGPLGHAGEESEVDN
DVDSPGSLRRGLRSTSYRRAVVSGFDFDSPTSSKKKNRMSQPVLKVVMED
KEKFSSLGRIKKKMLKGQGTFDGEENAVLYQNYKEKALDIDSDEESEPKE
QKSDEKIVIHHKPLRSTWSQLSAVKRKGLSQTVSQEERKRQEAIFEVISS
EHSYLLSLEILIRMFKNSKELSDTMTKTERHHLFSNITDVCEASKKFFIE
LEARHQNNIFIDDISDIVEKHTASTFDPYVKYCTNEVYQQRTLQKLLATN
PSFKEVLSRIESHEDCRNLPMISFLILPMQRVTRLPLLMDTICQKTPKDS
PKYE VC KR ALKE VS KLVRLCNEG ARKMERTEMM YTINS QLEFKIKPFPL V
SSSRWLVKRGELTAYVEDTVLFSRRTSKQQVYFFLFNDVLIITKKKSEES
YNVNDYSLRDQLLVESCDNEELNSSPGKNSSTMLYSRQSSASQSPLYSDS
P*

(In protein sequences * denotes a stop codon is present at this location of a coding sequence.)

Another example of an SGEF protein prototype is about 446 a.a. in length. A 446 a.a. protein preferably has the following sequence:

(SEQ ID NO 2.)
MDGESEVDFSSNSITPLWRRRSIPQPHQVLGRSKPRPQSYQSPNGLLITD
FPVEDGGTLLAAQIPAQVPTASDSRTVHRSPLLLGAQRRAVANGGTASPE
YRAASPRLRRPKSPKLPKAVPGGSPKSPANGAVTLPAPPPPPVLRPPRTP
N AP APCTPEEDLTGLT ASP VPS PT ANGL A ANNDS PGS GS QS GRKAKDPER
GLFPGPQKSSSEQKLPLQRLPSQENELLENPSVVLSTNSPAALKVGKQQI
IPKSLASEIKISKSNNQNVEPHKRLLKVRSMVEGLGGPLGHAGEESEVDN
DVDSPGSLRRGLRSTSYRRAVVSGFDFDSPTSSKKKNRMSQPVLKVVMED
KEKFSSLGRIKKKMLKGQGTFDGEENAVLYQNYKEKALDIDSDEESEPKE
QKSDEKIVIHHKPLRSTWSQLSAVKRKVILIVGFMEMKDGRLRGGK*

Another example of an SGEF protein prototype is at least about 110 a.a. in length, likely longer. The at least 110 a.a. protein prototype preferably has the following N-terminal sequence:

(SEQ ID NO 3.)
MDGESEVDFSSNSITPLWRRRSIPQPHQVLGRSKPRPQSYQSPNGLLITD
FPVEDGGTLLAAQIPAQVPTASDSRTVHRSPLLLGAQRRAVANGGTASPE
YRAASPRLRR (An incomplete sequence, the mRNA
does not comprise a stop codon at this location.)

It will be noted that the above prototype sequences comprise the same amino acid sequence at their N-termini.

Another example of an SGEF protein is about 154 aa in length. A 154 aa protein prototype would preferably have the following sequence:

(SEQ ID NO 4.)
MKSLILLQGRTAPQCSIQDRALPVSHLFTLTVLSNHANEKVEMLLGAET
QSERARWITALGHSSGKPPADRTSLTQVEIVRSFTAKQPDELSLQVADV
VLIYQRVSDGWYEGERLRDGERGWFPMECAKEITCQATIDKNVERMGRL
LGLETNV*

Yet another example of an SGEF protein is about 137 aa in length. A 137 aa protein prototype would preferably have the following sequence:

(SEQ ID NO 5.)
MFCFLLEAQLVSLNAGPELQRKISKCTLLDCTCFFSATGNLVCPLLASAL
TQVEIVRSFTAKQPDELSLQVADVVLIYQRVSDGEWERSYGTLVVQDAEC
YRPEECHFVIIAHIPNLDMLMFEITYMYCLLISKAKP*

It will be noted that the protein sequences of SEQ. ID. NOs. 4 and 5 comprise an overlap region. Therefore, the protein of the invention is any of the above illustrated protein prototypes, as well as any other protein construct based on the SGEF gene sequence, which is at least about 90 amino acids, up to about 900 amino acids long. Such proteins result, for example, from alternative transcriptional promoters, alternative processing of the transcripts and/or alternative protein processing possibly mediated by specific 5′region enhancers or repressors.

The SGEF protein of invention does not have to be identical to a naturally derived SGEF protein, it can be a variant protein. Two amino acid sequences are said to be “identical” if the two sequences, when aligned with each other, are having exactly the same amino acid sequences, with no gaps, substitutions, insertions or deletions. The variant proteins of the invention are, preferably, identical to one of the prototype amino acid sequences identified by SEQ ID NOs 1-5.

However, the proteins of the invention do not have to be identical to any of these sequences. The scope of the invention includes protein variants having sequences that are “substantially identical” (as defined below) to one of the sequences identified by SEQ ID NOs 1-5, or to any protein based on the SGEF gene sequence.

The protein sequence of the invention may comprise acceptable substitute amino acids. Certain amino acids are “like” amino acids in certain aspects, e.g. size, shape, and polarity. “Like” amino acids substitutions and their use as substitutes are concepts well understood in the art. By way of example, glycine, alanine, serine, threonine and methionine are considered to be “short side chain” amino acids; isoleucine, leucine and valine are all hydrophobic in nature; asparagine and glutamine are polar; aspartic acid and glutamic acid are acidic; lysine, arginine and histidine are basic; and tyrosine, phenylalanine, and tryptophan have aromatic group shaped side chains. Such “like” substitutions do not likely have a significant impact on the protein's folding and function. In accordance to the invention, if the like-substitutions do not amount to changes in amino acid identity at the corresponding position in more than 60% of the protein sequence, the protein is an SGEF protein of the invention. Preferably, the like-aa substitution comprises less than about 60% of the sequence, more preferably about 50%, or 45%, or 40%, yet more preferably, about 35%, 30%, 25%, 20%, or 15% and more preferably yet, about 10%, 5% or about 0% like-amino acid substitutions.

In respect to the proteins identified by SEQ ID NOs 1-3, certain substitutions including V29L; L60S; F203S; L461M, S707T; Q743H and S744L; P745F are “acceptable substitutions” and their presence does not contribute to the above calculation of allowable substitutions. Likewise, substitutions including S25T; H26S and F28L are acceptable substitutions of the prototype sequence of SEQ ID No 4.

Single nucleotide polymorphisms (SNPs), have been frequently involved in controlling the level of expression of the gene in different tissues and to thus mediate predisposition to, as well as protection from, different disorders. Such SNP variants have been implicated in multiple disorders from breast cancer to diabetes and age-related macular degeneration. SNP variants of SGEF are important factors in mediating visual capacity via macular function, bi-manual and hand-eye coordination and speed via corpus callosum development, liver homeostasis and sensitivity to drugs, alcohol or toxic substances, the immune function relative to viral or bacterial pathogens and mounting of an inflammatory response manifesting as fever, interferon, interleukin and other inflammatory mediator synthesis or secretions. A SGEF protein variant, wherein the amino acid sequence is modified in correspondence to an SNP, are considered SGEF proteins desirable as therapeutic agent of diseases that correlate with development and function of the retinal macula, the corpus callosum, the hippocampus, the liver, the immune system, and is a factor in a fever response to infections or reduce the risk of development or arrest of certain cancers. Likewise, the SNP might beneficially reduce the level of gene expression. For example, it can reduce the likelihood of cancer, of inflammation and of arteriosclerosis by blocking trans-endothelial migration. Thus any natural variant of the SGEF gene or portion thereof can be advantageously expressed in a patient. The natural variant expressed is preferably a variant comprising a SNP variation in a functional domain of an SGEF protein. More preferably, the SNP causes a change in the primary structure of a protein domain such as the DH domain and the PH domain or the SH3 domain.

Moreover, a protein sequence need not be perfectly aligned to another sequence and be expected to retain functionality. Short gaps and additions are tolerated. A preferred protein of the invention has the same overall length as a respective prototype listed in one of the sequences of SEQ ID NO 1-5, but the sequence may be up to about 15% different in length, as long as any one deletion or insertion does not comprise more than 15 consecutive amino acid residues. Preferably, the difference in length of the sequences is about 12%, 10% or 8%. More preferably, the length differences are about 7%, 6%, 5%, 4%, 3%, 2%, or 1%. Preferably, an addition or deletion is no more than about 12, 10, 8, 7, 5, 3, or 2 consecutively strung out aa residues.

Moreover, the protein(s) of the invention (or gene construct corresponding thereto) must comprise at least one and preferably more than one of the functional domains listed below. The choice as to which domain(s) is/are included depends on the specific function desired of the SGEF of the invention. The choices become clear when the domain's role is considered.

The acronym GEF in SGEF stands for guanine nucleotide exchange factor which is the rate limiting step of the GTPase cycle, which is accelerated by the GEF. The protein also includes a C terminal SH3 protein domain, flanking the hinge and binding specificity loops which binds to proline-rich ligands. It is also referred to as the SRC Homology 3 domain. The SH3 is a small protein domain of about 60 amino acids residues. It has been identified in several other protein families such as: tyrosine kinases, phosphatases and cytoskeletal proteins like myosin 1, spectrin and contactin; PI3 Kinase, Ras GTPase activating protein (Ras-GAP), the GEF VAV, Crk adapter protein, CDC24 and CDC25. The SH3 domain has a characteristic beta-barrel fold which consists of five or six β-strands arranged as two tightly packed anti-parallel R sheets. The linker regions may contain short helices. The SH3 domain is usually found in proteins that interact with other proteins and mediate assembly of specific protein complexes like scaffolds, typically via binding to proline-rich peptides (specifically left handed type 1 polyproline helices that repeat every 3 residues in their respective binding partner. Many SH3-binding epitopes of proteins have a consensus sequence:

### -X-P-p-X-P-

-1-2-3-4-5-
with 1 and 4 being aliphatic amino acids, 2 and 5 always and 3 sometimes being proline. The sequence binds to the hydrophobic pocket of the SH3 domain. Interaction depends on hydrophobic contacts of proline with conserved hydrophobic residues in a shallow groove on the SH3 domain as well as hydrogen bonds with ligand peptide carbonyl oxygen. SH3 domains that bind to a core consensus motif R-x-x-K have been described. Examples are the C-terminal SH3 domains of adaptor proteins like Grb2 and Mona (a.k.a. Gads, Grap2, Grf40, GrpL etc.). Other SH3 binding motifs have emerged and are still emerging in the course of various molecular studies, highlighting the versatility of this domain. Preferably, the proteins of the invention are similar to prototypes identified by SEQ ID NOs 1 and 2 and contain the SH3 domain.

The SGEF proteins of the invention also may include up to four other domains upstream of SH3. Preferably, proteins similar to the prototype of SEQ ID NO 1 contain a DH domain.

The Dbl homology (DH) domain is an extended helical domain of more than 200 amino acid that binds nucleotide free GTPase (Aittaleb M. et. Al., Mol. Pharmacol 77(2): 111-25(2010) and is a Tiam1-Rac1 interaction site for the switches 1 and 2 regions of Rac 1 that mediate binding and release of GTP/GDP by Rac1 and thus constitute the main GTPase interaction site with its binding specificity loops. The DH domain is composed of a unique extended bundle of alpha helices. See pawsonlab.mshri.on.ca/index.php?option=com_content&task=view&id=154&Itemid=64, last visited on Feb. 15, 2011. It induces Rho family GTPases to displace GDP. It thus activates the Rho GTPase by allowing binding to GTP. Rho GEF is thus a strong activator of the Rho G family of GTPases by catalyzing the rate limiting step of the GTPase cycle. Rho GEF in turn activates the downstream effectors of RhoG like Rac1 via ELMO (a Dockl80 binding protein) see Katoh H. et al. Nature 424:461-64 (2003) and others like Cdc42 see Wennerberg K. S. M. et. al., J. Biol. Chem. 277: 47810-817 (2002).

The DH domain in the SGEF protein is followed by a pleckstrin homology (PH) domain. The Pleckstrin homology domain (PH domain) is a phosphorylation-sensitive protein adapter domain of approximately 120 amino acids or P-ephexin that functions as a protein-protein interaction site domain present in kinases (like BTK Bruton's tyrosine kinase), scaffolds, GEFs, GAPs, phospholipase C delta, and dynamin. The PH domain binds polyphosphoinositides, like PIP2 and PIP3, which target the protein to membrane bilayers rich in PIPs, which are synthesized when receptor tyrosine kinases RTK or G protein-coupled receptors GPCR activate phosphoinositide 3 kinase (PI3K). It constitutes the SGEF hinge region which is a proline-rich region binding site with a PH fold which permits target cell location and interaction with a binding protein, it is a common domain to signaling proteins and binds inositol phosphate to target proteins (Marchler-Bauer, et al., Nucleic Acids Res. 37:(D)205-10 (2009). PH domains occur in a wide range of more than 200 proteins involved in intracellular signaling or as constituents of the cytoskeleton. Baltimore D, et al. Cell 73 (4): 629-630(1993); Hemmings B. A., et. al, Nature 363(6427):309-310 (1993); Gibson T, et al, Trends Biochem. Sci. 18 (9):343-348(1993); Gibson T J, et al. Trends Biochem. Sci. 19 (9): 349-353 (1994); Pawson T., Nature 373(6515):573-580 (1995); Hemmings B. A and Ingley E. J., Cell. Biochem. 56(4):436-443 (1994). While not absolutely required for catalysis of nucleotide exchange, the PH domain appears to greatly increase catalytic efficiency in many cases.

The SGEF N terminal proline-rich domain is another domain which may be included. It promotes protein-protein interaction, of the intra-molecular type, that may inhibit Rho GEF activity and may interact with the SH3 domain and maintain SGEF in the inactive state unless stimulated by specific stimuli. Zheng, Y. Trends Biochem Sci. 26:724-732 (2001) and Macias M J et al, FEBS Lett. 513:30-37 (2002).

The two nuclear localization signals of SGEF are another feature desirably present in the SGEF of the invention to achieve specific effects. They provide the possibility the SGEF can translocate to the nucleus when stimulated by specific signals like the VAV1 GEF which translocates to the T-cell nucleus. Clevenger C. V. et al., J. Biol. Chem. 270: 13246-13253 (1995).

The protein prototype of SEQ ID NOs 1 and 2 contain a vacuolar domain. The protein prototype of SEQ ID NO 4 contains an SRC homology 3 domain and a variant SH domain.

SGEF “protein variant” or “protein of the invention” are used interchangeably in the description of the invention.

In accordance to the invention, a functional SGEF protein is provided. For a SGEF protein, “functional” means the protein is provided essentially intact and is substantially similar to a prototype protein as described above. “Functional” alternatively means that the SGEF protein of the invention functions substantially similar to the natural SGEF enzyme or a prototype SGEF enzyme, in a functional assay for the designated function required for application of the present invention.

An example of an assay that would compare a SGEF protein of the invention with a natural SGEF or a SGEF prototype SGEF would be the Cell Biosciences Firefly 3000 Protein Analysis System. The Cell Biosciences Firefly 3000 Protein Analysis System quantifies the phosphorylation of signaling proteins. Relative changes in the content of total and GTP-bound Rho G-proteins which are a reflection of SGEF protein activity can be quantified by Western immunoblot and GTP-binding ELISA or preferably by assaying myc-tagged RhoG. Katoh H et al. Nature 424:461-464 (2003). Another assay example would be Brefeldine A, which can be used to block nucleotide exchange on some Arfs catalyzed by GEFs, disrupting membrane traffic between Golgi complex and endoplasmic reticulum. “Functions substantially similar” in this context means it has at least about 50% of the activity of the natural or prototype SGEF, preferably at least about 60%, and yet more preferably it has at least 70% or higher, up to about two times the activity of the natural or prototype SGEF. The substantially similar protein has, at a minimum, a primary aa. sequence structure as limited above, i.e., it has no more, and preferably less than 60% like-a.a. substitutions and, preferably, less than 60% non like-a.a. substitutions. Preferably, in addition, the substantially similar protein is, at most, 15% different in length from its prototype protein (and preferably less than 15%), as long as any one deletion or insertion does not comprise more than about 15 consecutive amino acid residues, and preferably less than 15 a. a. residues. See above. More preferably yet, the protein includes the domains of its prototype SGEF protein, in the same order and substantially similarly spaced (the distance between domains, if they are present, does not differ by more than about 15% from the distance between the same domains in the prototype).

The methodologies described herein for measuring SGEF levels or activities serve also as a diagnostic assay, wherein a departure from normal levels of at least about 20%, more preferably at least 30%, 40%, 50% or more, up to about 400%, is indicative of a SGEF related disease state.

In accordance to another aspect of the invention, a SGEF gene, as well as an expression vector is provided. The SGEF gene is functional. “Functional” means here that the gene comprises a coding region corresponding to an SGEF protein. Preferably, the SGEF gene expresses an SGEF protein of the invention, as defined above. Functional also means that the gene and vector are constructed so the SGEF protein of the invention is expressed in the target cell, tissue or organism.

The gene is, preferably, constructed from a cDNA (i.e., sans introns). However, any manner of presenting an accurate template for SGEF expression is within the scope of the invention, including RNA template or genes comprising one or more exons, as long as the system allows for expression of a functional SGEF gene-derived protein. The expression of the SGEF protein can be in any system. For example the expression is in bacterial cultures, yeast cultures, bacculoviruses, or, preferably, in a plant system or a mammalian cell or tissue culture. More preferably, the gene is engineered for expression in a mammal, in vivo. If delivered to a target mammal, the expressed protein is delivered directly, without the need of purification and formulation (see below). Preferably the mammal to which the SGEF gene or purified protein is provided is a human. Methods of engineering the gene for expression are well known in the art. Useful vectors are well known. Examples of useful expression vectors include the AAV adeno associated virus of which many types are known with specific organ, tissue or even cell-type specific targeting efficiency or retroviral vectors or any other vectors enabling the cargo gene cDNA or RNA to enter and be efficiently expressed in the target cell, tissue, organ or organism. Jakovcevski, M. et. al, Cold Spring Harb. Protoc. (4):5417 (2010) also using an appropriate targeted promoter.

It will be recognized that the gene might comprise various desirable features and substitute features as understood by a skilled artisan. By way of example only, the gene might be under the control of features unlike the features in the natural gene. These might include, for example, different promoters (for example the chicken beta actin promoter) or regulated promoters, or different 5′ and 3′ UTRs specific enhancer motifs and alternative polyadenylation signals, if any.

The gene does not have to mimic in nucleic acid sequence the natural SGEF gene or relevant portions thereof, as long as it encodes for an SGEF of the invention, i.e. for the functional SGEF protein prototypes or substantially similar and functional proteins thereof, some of which are described above. Single nucleotide polymorphisms (SNPs) have been frequently involved in controlling the level of expression of the gene in different tissues and to thus mediate predisposition to as well as protection from different disorders. Such SNP variants have been implicated in multiple disorders from breast cancer to diabetes and age-related macular degeneration. SNP variants especially those involving enhancers or repressors of SGEF are important factors in mediating visual capacity via macular function, bi manual and hand-eye coordination and speed via corpus callosum development, liver homeostasis and sensitivity to drugs, alcohol or toxic substances as well as level of immune function relative to viral or bacterial pathogens and mounting of an inflammatory response manifesting as fever, interferon, interleukin and other inflammatory mediator synthesis or secretions. The gene might comprise alternative codons, cryptic open reading frames within introns or other features, such an ORF15 of the RPGR gene, which, like SGEF, is a GTPase regulator, but is a member of the rab family. Yokoyama, A., Am. J. Med. Genet. 104(3):232-8 (2001).

Since leukocyte transendothelial migration is critical to the important physiologic processes of immune surveillance and inflammation, and since SGEF or RhoG inhibition has been shown to inhibit transendothelial formation (van Buul et. ah, J. Cell Biol. 178(7): 1279-93 (2007)), lack of fever and the lack of inflammatory response observed in our patient lacking SGEF protein confirms the key role of SGEF in mounting an immune and a normal inflammatory response.

The effect of the missing SGEF protein product on the severe pathogenicity of the Epstein-Barr (“EB”) virus is a key to the mechanism of the human pathogenicity of the EB virus, causing not only infectious mononucleosis but also chronic active EBV infections, Hemophagocytic lympho-histiocytosis, nasopharyngeal carcinomas and Burkitt's lymphoma in certain African populations. This implies that the said SGEF protein product is a key factor for the human immune system to mount a response to the EB virus. This interaction of the EB virus with the SGEF protein structure is thus a useful tool to design a specific treatment against the EB virus infection and thus not only against infectious mononucleosis but also against chronic forms of EBV infections and neoplastic complications such as Burkitt's lymphoma and possibly other lymphomas. Kanno, H. et. al, Clin Exp. Immunol. 2008 Mar. 151(3):519-27. Epub (2008).

SGEF is a key factor in defense against EB virus infection as shown in the SGEF deficient proband, who could not mount a response to the infection for months and developed very severe liver complications of the disease. We are not implying here a mechanism of direct or indirect of EB virus interaction with the SGEF protein but simply giving evidence that since SGEF protects against EB virus infection, its expression is a treatment against both infectious mononucleosis and its neoplastic complications such as Burkitt's lymphoma.

Guanidylate binding proteins 1 and 5 over-expression has been shown to be associated with chronic active EB virus infection by microarray analysis. Ito Y, Infect Dis. 197(5):663-6 (2008). This shows that excessive guanidylate binding depletes GDP and blocks the activity of SGEF by depleting its substrate leading to chronic EBV disease.

The method of delivery of the SGEF gene or protein is not limiting to the invention. An artisan skilled in the art will choose between any method available and convenient. For example, gene delivery might include various viral vectors. Protein delivery might include liposomes or formulated solid or liquid preparations, suitable for injection or alimentary canal delivery, or patches or suppositories, or eye drops or nasal drops or topical treatments etc. Preferably the delivery system provides systemic delivery, such as might be preferentially achieved by delivery directly to blood, the circulatory system.

Various techniques can be used to deliver the target protein to membrane proximity where they can be functional such as but not exclusively using Polyethylene glycol (PEG) chains conjugated to liposomes through a disulfide bond cleavage site to improve intravascular circulation time. Filamentous micelles made from PEG copolymers and either non-degradable polyethylene or degradable polycaprolactone. Trans-activating transcriptional activating TaT peptide incorporation can engage macropinocytosis for cell entry while use of ligands such as folic acid, transferrin or cholesterol can facilitate uptake through caveolin-mediated endocytosis. Protein cargos can thus be targeted to the liver or other organs, to the cytosol. Vasir J. K., et. al, Adv. Drug Deliv. Rev. 59(8):718-28 (2007); Zhang Z., et al, Angew Chem. Int. Ed. Engl. 48(48):9171-5 (2009); Hodoniczky J., et. al, Biopolymers, 90(5):595-603 (2008); and Misra R. and Sahoo, S. K., Eur. J. Pharm. Sci. 39(1-3): 152-63 (2010). The protein can be targeted to prostate or other tumors using the enhanced permeability and retention effect of tumors or peptide ligands. Petros R. A., et al, Nat. Rev. Drug Discov. (8):615-27 (2010).

More preferably direct delivery of the transgene packaged into a viral vector such as into the eye via sub-retinal or intravitreal injections or by iontophoresis using a magnetic field to deliver the nucleic acid directly to the retina or other specific part of the eye. Even more preferably direct delivery to specific parts of the brain like the hippocampus by a viral vector cargo including a channel rhodopsin (ChR2) variant or the like and an inactive SGEF transgene which is then activated in targeted brain sites using low intensity light possibly via a LED or similar light device. Diester I. et al, Nat Neurosci. 14(3):387-97 (2011); LaLumiere, R. T., Brain Stimul. 4(1): 1-6 (2011); Grossman N., et. al, IEEE Trans Biomed Eng. (2011). February 14:[Epub ahead of print].

While it is possible for a compound of the invention to be administered alone, it is preferable to administer the compound as a pharmaceutical formulation comprising also pharmaceutical carriers, resulting in a composition. The pharmaceutically acceptable compositions of the invention comprise one or more compounds as an active ingredient in admixture with one or more pharmaceutically acceptable carriers and, optionally, one or more other compounds, drugs, ingredients and/or materials. Regardless of the route of administration selected, the compounds of the present invention are formulated into pharmaceutically acceptable dosage forms by conventional methods known to those of skill in the art. See, e.g., Remington's Pharmaceutical Sciences, Mack Publishing Co., Easton, Pa.

The scope of the invention also includes control of diseases caused by over-expression of SGEF or where reduced SGEF expression, often locally, improves disease outcome or prognosis. Although the mechanism of action does not limit the scope of the invention, it is proposed that the over-expressed SGEF might interact with the cytoskeleton to mediate cell movement, or the over-expressed SGEF might affect cell-cell interactions, transendothelial migration, cell division and multiplication, as well as cell transformation. The short C terminal isoform cSGEF is sensitive to androgen in prostate cancer cells. Qi, H. et al, Endocrinology 144:1742-1752 (2003). Furthermore, SGEF expression in prostate cancer cells is activated by TOP2B (topoisomerase 2B). Haffner, Michael C, et al, Nature Genetics 42:668-675 (2010). The role of SGEF activation of RhoG (which in turn activates Rac) is a factor in breast cancer cell migration. Hiramoto et al, J Cell Biol. August 9, 190(3):461-77 (2010), Epub 2010 Aug. 2. Furthermore, RhoG overexpression has been shown as one of the downstream effects of the pituitary tumor-transforming 1 PTTGl/Securin in human oseophageal squamous cell carcinoma to increase cell motility and lymph node metastasis. Yan, S. et al, Cancer Res 69(8): 3283-3290 (2009).

Because of the pivotal role of SGEF in cell multiplication and growth, we conclude that the prolonged excess or activation of SGEF gene expression is a factor in carcinogenesis, metastasis and cell transformation. Inhibitors of SGEF likely play a role in cancer treatments of prostate, breast, oesophageal, gastro-intestinal and other cancers. Particularly likely cancer or tumors that are caused by SGEF over-expression are in the brain, prostate, eye, breast, esophagus, cervical and liver. SGEF inhibition will likely be efficient in blocking metastasis. Alternatively, under expression of SGEF may be responsible, and require correction/overexpression, in the treatment of other cancers, e.g. ovarian cancers. Yet further, under expression of SGEF may need to be corrected to improve chemotherapy outcomes. Overall, in many medical conditions, the need is not between extremes of no SGEF expression or particularly large over-expression, but, rather, a more controlled, nuanced expression levels.

A proper treatment transiently and/or to a limited extent reduces the level of SGEF. Preferably, the SGEF levels would be controlled in specific tissues that are suspect or known to be undergoing tumor generation or evidence of possible cancer formation. The level of SGEF can be controlled by any means known in the art. For example, a specific anti-SGEF antibody is provided. Methods to develop antibodies are known, including humanized antibodies, single chain antibodies, ab2s, etc. Alternatively, the SGEF gene expression is inhibited by providing anti sense RNA (sRNA) siRNA (small interfering RNA), sh RNA (short hairpin RNA) such as p.Sec.shRNA from plasmid, gene traps or ribozymes. The anti SGEF agent delivery is preferentially provided in a tissue specific manner. Specific tissue delivery methods are known. For example, the vector might have affinity to tissue specific markers. In particular Adeno-Associated Virus viral vectors are a prime example where different subtypes, AAV2a, based vectors for example are specifically targeting different tissues or tissue compartments of the eye. For example, delivering locally to the eye using PLGA nanoparticles, or using sodium alginate such as in 10% solution in phosphate-buffered saline which will increase remanence as well known to those of the art. Kotagale et. al, Indian J Pharm Sci., 72(4):471-9 (2010).

The anti SGEF agent is preferentially provided to a patient where the SGEF expression level is high. That can be measured by quantitative Elisa assays, or PCR assays and so forth, as well known by artisans skilled in the art.

Any of SGEF gene or protein or anti-SGEF agent will require delivery into the mammal and formulation for optimal delivery. The formulation choices will match the desired delivery channel and be consistent with delivery of nucleic acid for protein expression or purified protein delivery. Which carrier agents to use will be a matter of choice, but a proper pharmaceutical carrier or expedient must be chosen to improve at least one from among solubility, stability and bioavailability in vitro and in vivo, and enhance delivery of the therapeutic agent so as to maximize absorption and delivery to the target cell, tissue, organ or organism and such as to minimize toxicity and allergy risks. Artisans skilled in the art will know how to choose the appropriate carrier agent(s).

Pharmaceutically acceptable carriers are well known in the art and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, nanoparticles or charged nanoparticles, gels such as sodium alginate, oils (e.g., corn, germ, olive, castor, sesame, cottonseed, and groundnut), cocoa butter, waxes (e.g., suppository waxes), paraffins, silicones, talc, salicylate, etc. Each pharmaceutically acceptable carrier used in a pharmaceutical composition of the invention must be “acceptable” in the sense of being compatible with the other ingredients of the formulation and not injurious to the subject. Carriers suitable for a selected dosage form and intended route of administration are well known in the art, and acceptable carriers for a chosen dosage form and method of administration can be determined using ordinary skill in the art. See, e.g., Remington's Pharmaceutical Sciences, supra, and The National Formulary (American Pharmaceutical Association), Washington, D.C. and Handbook of Pharmaceutical Excipients 6th edition (2009) Edited by Raymond C. Rowe, Paul J. Sheskey and Marian E. Quinn., Development Editor, Royal Pharmaceutical Society, UK.

The pharmaceutical compositions of the invention may, optionally, contain additional ingredients and/or materials commonly used in such pharmaceutical compositions. These ingredients and materials are well known in the art and include fillers or extenders, such as starches, lactose, sucrose, glucose, mannitol, and silicic acid; binders, such as carboxymethylcellulose, alginates, gelatin, polyvinyl pyrrolidone, hydroxypropylmethyl cellulose, sucrose and acacia; humectants, such as glycerol; disintegrating agents, such as agar-agar, calcium carbonate, potato or tapioca starch, alginic acid, certain silicates, sodium starch glycolate, cross-linked sodium carboxymethyl cellulose and sodium carbonate; solution retarding agents, such as paraffin; absorption accelerators, such as quaternary ammonium compounds; wetting agents, such as cetyl alcohol and glycerol monosterate; absorbents, such as kaolin and bentonite clay; lubricants, such as talc, calcium stearate, magnesium stearate, solid polyethylene glycols, and sodium lauryl sulfate; suspending agents, such as ethoxylated isostearyl alcohols, polyoxyethylene sorbitol and sorbitan esters, microcrystalline cellulose, aluminum metahydroxide, bentonite, agar-agar and tragacanth; buffering agents; excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; inert diluents, such as water or other solvents; preservatives; surface-active agents; dispersing agents; control-release or absorption-delaying agents, such as hydroxypropylmethyl cellulose, other polymer matrices, biodegradable polymers, liposomes, microspheres, aluminum monosterate, gelatin, and waxes; opacifying agents; adjuvants; wetting agents; emulsifying and suspending agents; solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; propellants, such as chlorofluorohydrocarbons and volatile unsubstituted hydrocarbons, such as butane and propane; antioxidants; agents which render the formulation isotonic with the blood of the intended recipient, such as sugars and sodium chloride; thickening agents; coating materials, such as lecithin; and sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

Pharmaceutical compositions suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, powders, granules and the like) may be prepared by mixing the active ingredient(s) with one or more pharmaceutically-acceptable carriers and, optionally, one or more fillers, extenders, binders, humectants, disintegrating agents, solution retarding agents, absorption accelerators, wetting agents, absorbents, lubricants, and/or coloring agents. Solid compositions of a similar type maybe employed as fillers in soft and hard-filled gelatin capsules using a suitable excipient. A tablet may be made by compression or molding, optionally with one or more accessory ingredients. The compositions may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form such as microbeads.

Liquid dosage forms for oral administration include pharmaceutically-acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

Pharmaceutical compositions for rectal or vaginal administration may be presented as a suppository, which may be prepared by mixing one or more active ingredient(s) with one or more suitable nonirritating carriers which are solid at room temperature, but liquid at body temperature and, therefore, will melt in the rectum or vaginal cavity and release the active compound. Pharmaceutical compositions which are suitable for vaginal administration also include pessaries, tampons, creams, gels, pastes, foams or spray formulations containing such pharmaceutically-acceptable carriers as are known in the art to be appropriate.

Dosage forms for the topical or transdermal administration include powders, sprays, ointments, pastes, creams, lotions, gels, solutions, patches, drops and inhalants. The active compound may be mixed under sterile conditions with a suitable pharmaceutically-acceptable carrier. The ointments, pastes, creams and gels may contain excipients. Powders and sprays may contain excipients and propellants.

Pharmaceutical compositions suitable for parenteral administrations comprise one or more compound in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption. They can be formulated to be administered intravenously, intra peritoneally, intra thecally, intraocularly (such as subretinally, intravitreously or others) and injected into any organ or vessel.

In some cases, in order to prolong the effect of a drug, it is desirable to slow its absorption. This may be accomplished by the use of a liquid suspension of crystalline or amorphous material having poor water solubility.

The rate of absorption of the drug then depends upon its rate of dissolution which, in turn, may depend upon crystal size and crystalline form. Alternatively, delayed absorption of a parenterally-administered drug may be accomplished by dissolving or suspending the drug in an oil vehicle. Injectable depot forms may be made by forming microencapsulated matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter.

The drug could also be contained into a medical device container and administered for slow release into the target organ (such as located in the eye) or any organ or tissue or coated onto an appropriate medical device such as located in blood or other vessels, any organ or tissue.

In some cases transgene expression has been activated in specific tissues like the brain by remotely turning on a local LED activating a chromoprotein moiety like channelrhodopsin or other variously engineered chromoproteins, which switches on the gene of interest as described above. The formulations may be presented in unit-dose or multi-dose sealed containers, for example, ampoules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

The SGEF protein of the invention may be delivered directly, i.e. in a protein form or indirectly, as a gene system for expression of the SGEF protein in a mammal. More preferably, more than one SGEF variant is delivered to the mammal. Preferably, the mammal is a human.

More preferably the mammal, prior to the SGEF variant(s) delivery has been diagnosed as having multiple deficiencies from among clinical or subclinical condition or predisposition for a disease associated of at least the structure or function of macula, hippocampus function or development, liver disorder, and immune function disorders.

Disorders of the macula include, for example, retinal disorders, macular disorders, macular dystrophies or macular degenerations like age-related macular degeneration, geographic atrophy, diabetic retinopathy, glaucomatous retinal or optic nerve dysfunction or any other visual disorders such as but not exclusively Stargardt's disease, Best's disease, albinisms of all types, Daltonism, achromatopsias of all types, retinoschisis, cone or rod disorders such as but not exclusively retinitis pigmentosa of all types or cone-rod dystrophies.

Disorders of the corpus callosum including hypoplasia or absence or even thickened corpus callosum involving right-left hand, foot or general coordination disorders, including bi manual coordination and hand-eye coordination disorders, common in visually impaired persons. Mueller K L et. al, Behav. Neurosci. 123(5): 1000-11 (2009).

Hippocampus development deficiency would include, for example, any type of memory or cognitive dysfunction such as intellectual deficiency, mental retardation but not exclusively post-traumatic shock, post-blast or other brain injury or senile memory disorders (Di Stefano G. et al, Rejuvenation Res. 28 (2010)); spontaneous or medication or drug induced or any other degenerative brain disorders such as Alzheimer disease (Gomez Ravetti M, et. al, PLoS One. 13; 5(4):e10153 (2010) PMID:20405009 [PubMed—in process]); and any other brain dysfunction involving memory, or other brain dysfunctions such as Down syndrome, Attention Deficit Hyperactivity Disorder or William's syndrome or any other syndrome, illness or disease involving memory deficiency. Kuzumaki N., et. al, Synapse 64(8):611-6 (2010) and Paban V. et. al, Neurobiol. Learn. Mem. 94(1):42-56 (2010).

Liver disorders would include, for example, hepatitis (viral such as caused by A, B, C, Delta, EBV, CMV or other viruses; bacterial, fungal or parasitic infections), other liver disorders or infection of liver or gall bladder (such as gall bladder agenesis or biliary tract agenesis), other congenital liver diseases like Gilbert's disease, Crigler-Najjar disease, Tuftsin deficiency, cystic fibrosis, alpha1 antitrypsin deficiency, any type of glycuro-conjugation disorder such as jaundice of various origin such as but not exclusively newborn jaundice, kernicterus, liver cirrhosis of toxic, drug or ethanol intoxication origin and any other form of hepato biliary dysfunction such as liver steatosis of any origin or cholestasis of any origin.

Immune function disorders could be innate or acquired and would include, for example, immune deficiency disorders linked to HIV infection, congenital immune deficiencies such as SCID (severe combined) ADA (adenosine deaminase) deficiency, steroid induced immune deficiency and deficiency of any type such as but not exclusively like any type of viral, bacterial, fungal or parasitic infection or scepticemia or gram negative or any other septic choc or toxic shock syndrome or macrophage activation disorders etc.

The SGEF alone might be used to treat such disorders or infections or as adjuvant therapy in conjunction with other existing or future accepted therapies such as but not exclusively antibiotic or antiviral therapy, interferon or other such therapy.

Alternatively the mammal receiving the therapeutic or prophylactic treatment has been shown to have a defect in the SGEF gene. In another alternative, the mammal is a member of a family where one member of the family has been diagnosed as having one of the multiple deficiencies from among clinical or subclinical condition or predisposition for a disease associated of at least the structure or function of macula, corpus callosum, hippocampus, lack of fever, liver or immune function not exclusively as listed above. Alternatively, a member of the family has been shown to carry a mutation in the SGEF gene.

In accordance to an aspect of the invention, if any member of a family is shown to carry a mutation in the SGEF gene, other members of the family should receive SGEF therapy, whether they have displayed yet symptoms of defects in any of the organs or functions associated with SGEF defects in accordance to the invention. Correspondingly, members of a family where one individual has any of the SGEF-associated defects should be screened for defects in the SGEF gene.

The methods of gene defect screening are well known in the art. They involve such methodology as gene sequencing, nucleic acid hybridization, PCR analysis, high throughput sequencing also called next generation sequencing or detection of SGEF protein by assays (e.g. Elisa assays) with one or more antibodies specific for SGEF. Preferably, the defect is located within the coding region of a SGEF protein variant. More preferably, the mutation aborts anyone of the functions or the expression of a significant portion of the SGEF protein, at least about 2% of the protein variant, preferably a larger portion of the protein, more preferably it impairs a protein domain, from among SGEF protein domains listed above.

Because SGEF has a critical role in maintenance of certain organs, a prophylactic role is apparent now also in the handling and storage of tissue for transplantation. At a minimum, SGEF protein variants are advantageously given as part of the storage treatment and rehydration or preimplantation treatment at least of ocular tissue implants as well as liver transplants.

### Example 1. A Family Displays Evidence of Genetically Co-Transmitted Congenital Corpus Callosum Agenesis or Hypoplasia, Early Development Macular Dystrophy and Dysfunction, Liver and Immune Dysfunction

The proband is the second of three children, with two siblings, born to consanguinous first cousin parents. A normal 46 XX karyotype on fetal amniocytes was reported. The child developed congenital nystagmus and strabismus by 6 months. Brain ultrasound showed enlarged subarachnoid fluid spaces (also called external hydrocephalus) with normal cerebral ventricules and complete corpus callosum agenesis. Ophthalmologic examination at the age of 5 and a half years showed distance vision was about 20/200 with near vision of level 4 of Parinaud scale and on fundus examination, bilateral oval lesions of complete severe macular dystrophy with normal vessels and small optic papilla without pigmentary deposits. Patient had no photophobia, night blindness or obvious dyschromatopsia. Severe macular dystrophy was confirmed on angiogram and without increased auto-fluorescence, initial electroretinograms (ERG) were within normal limits and visual evoked potentials (“VEP”) showed absent pattern VEP at 15 and 30′ in favor of macular bundle dysfunction. Color vision testing using Farnsworth Munsell 15 hue test showed multiple type 3 errors compatible with macular dysfunction. Retinal Optical Coherence Tomography (OCT) examination showed poorly differentiated macular area with absent foveal pit, thinning of the retina and interruption of the photoreceptor layer. Center Macular thickness was 102μη at right and 92μη at left (50% of normal), right macular volume was 4.65 mm³ and 4.32 mm³ at left. Brain MRI confirmed complete corpus callosum agenesis and showed bilateral hippocampi hypoplasia. Fiber crossing mode MRI confirmed the complete absence of midline crossing callosal axonal fibers.

Patient's development, growth, intelligence and neurologic examination were assessed as normal. Child initially showed learning difficulties in first grade with slow memory and difficulties with graphic and visual memory tasks but was able to learn to read and write and entered 2nd grade after appropriate specialized intensive rehabilitation in speech and writing, psychomotor and orthoptic tasks.

Patient developed daily afternoon fatigue and sleeping attacks during school hours and early in the evening for 4-5 months. There was no evidence of seizures on repeated electro-encephalograms. Liver function tests were found to be abnormal with ASAT and ALAT liver enzymes levels at about 20 times normal values showing severe liver cytolysis 4 months after onset of illness without ever showing any sign of fever. Positive serology for both Epstein-Barr virus IgG and IgM as well as group A beta streptococcus were identified. The unusually protracted course of these two concurrent illnesses involving both viral and bacterial illnesses and the overall status and severity of the liver condition indicates immune dysfunction and liver homeostasis disturbance.

Congenital macular dystrophy diagnosed within the first year of life has not been previously described and thus represents a new form of developmental macular dystrophy.

Family history showed both parents had 20/20 vision but a granular texture was noted on maternal fundi. Both parents have a normal ERG, VEP retinal OCT and maternal multifocal ERG and brain MRI were normal while the father's multifocal ERG showed unilateral foveal macula dysfunction and his brain MRI showed non-specific small high-intensity white matter signals on T2 imaging.

The proband's older sibling is healthy with normal development and with 20/20 vision bilaterally with a normal fundus examination and a mildly abnormal brain MRI showed slight thinning at the junction of the posterior third of the corpus callosum. VEP, ERG and retinal OCT were unremarkable but multifocal ERG showed bilaterally abnormal foveal macular cone function with decreased activity: objective evidence of subclinical foveal cone dysfunction bilaterally. This is the first description of a congenital macular dystrophy which can appear with a congenital nystagmus or subclinical as in the older sibling. It can be part of an obvious corpus callosum and hippocampi anomaly with immune dysfunction as seen in the proband or with little neuroimaging or foveal anomaly like in the older sibling.

The gene defect associated with this description represents the cause of a novel neuro-ophthalmic autosomal recessive syndrome with congenital macular dystrophy, corpus callosum agenesis, hippocampus hypoplasia and immune dysfunction. This suggests the putative genetic cause of this disorder might involve the control of macular development as well as corpus callosum fiber neuronal guidance, hippocampus development, immune function, fever and inflammation.

### Example 2. The Genetic Locus for the Syndrome Described in Example 1 is Different from the Known Loci Responsible for Macular Dysfunctions

The syndrome described for the proband in Example 1 is different from the syndrome described by Descartes et al. in two siblings who, besides agenesis of corpus callosum and macular dystrophy also have dysmorphism, mental retardation and deafness. The DNA form this family was checked for the presence of the genetic defects responsible for the syndrome observed by Descartes et al. for mutation in the SGEF gene, with negative results. Accordingly, the malady of the family of Example 1 is of a different genetic causation relative to known genetic basis for syndromes involving macular dysfunctions. Descartes et al., Clin. Dysmorphol. 18(3): 178-80 (2009).

### Example 3. The Genetic Locus Responsible for the Syndrome of the Family in Example 1 is Identified

The 3q25.2 small 114 kb homozygous deletion involving 5 probes was identified in the proband by Array Comparative Genome Hybridization CGH performed on peripheral blood lymphocytes using the Agilent 105k platform (see FIG. 1) and confirmed by real time PCR assay. The deletion was then confirmed in the 2 siblings (as the same homozygous deletion) and in the parents (as the same heterozygous deletion) by quantitative PCR. Using build NCBI 36/hgl8 of the NCBI genome map, the deletion was found to involve a single gene and to remove the upstream 5′ region and the first 6 coding exons up to the 6th intron (see FIG. 2) of the Refseq SGEF (reference sequence) gene. See FIG. 1 for illustration of the Array CGH Agilent analysis of the deletion extent and location. The upper panel presents the comparative Genome Hybridization (CGH) analysis. The genomic DNA is cut into multiple fragments (cc 105,000 fragments) and then hybridized to specific probes matching specific, spaced out fragments of the genome. The probes and their relative location are indicated as dots. Five probes did not hybridize (appearing as the five dots displaced downwards). They indicated that the deleted DNA is within the 3q25.2 locus. The lower panel is a depiction of the SGEF genetic locus. The single line illustrates introns. The boxes illustrate exons. The arrow under the panel indicates the deletion area.

As it can be seen, five DNA probes (shown as small circles deviating from baseline levels) correspond to the homozygously deleted genomic DNA from the probed. The lower panel is a schematic representation of the region, where the single lines represents introns and the boxed regions represent exons.

The details of the probes used to delineate the exact position of the deletion are described in Table 2 and are based on NCBI build 36/hgl8 of the NCBI human genome map.

TABLE 2

| | Cyto | | | |
|---|---|---|---|---|
| Location of | genetic | Probes (from | Start site | Stop site |
| the Probe | Band | Affymetrix) | location | location |
| Last normal | 3q 25.2 | A_14_P137770 | 155170885 | 155170944 |
| First deleted | 3q 25.2 | A_16_P16459148 | 155239440 | 155239499 |
| Last deleted | 3q 25.2 | A_14_P136564 | 155353325 | 155353384 |
| Next normal | 3q 25.2 | A_16_P16459487 | 155368151 | 155368210 |

The minimal size of the deletion is 113944 base pairs and its maximum size is 197207 base pairs. Accordingly, the gene transmitted within the family of the Example 1 is an SGEF gene. The syndrome was caused by a homozygous deletion within that gene.

### Example 4. The Segregation Analysis of Example 1 and the Identification of the Gene Defect Lead to Conclusions as to the Role of SGEF Gene

From the segregation analysis of Example 1 and the gene mapping data of Example 3, we conclude that the SGEF homozygous deletion is sufficient to also cause subclinical phenotypes like the bilateral foveal macular dysfunction and minimal corpus callosum development defect seen in the brother who does not harbor the double ABCA4 variant. The cumulative effect of SGEF homozygous deletion with the double ABCA4 variant possibly causes the added phenotype observed in the proband with congenital nystagmus with macular dystrophy but it could also be linked to SGEF effect alone and other causes like epigenetic factors or other genetic factor. Indeed the non-ocular phenotype seen in the proband with complete agenesis of the corpus callosum, the hippocampal hypoplasia and the immune dysfunction is difficult to attribute to such mild effect ABCA4 variants. Bhongsatiern J, et al, J Neurochem. 92(5): 1277-1280 (2005); Tachikawa M, et al, J. Neurochem. 95(1):294-304 (2005); Warren M S et al, Pharmacol Res. 59(6):404-13 (2009); and Tsybovsky Y. et al, Adv Exp Med Biol. 703:105-25 (2010).

Therefore the variable severity of the ocular and brain phenotype between proband and older sib could possibly be due to the redundancy of the GTPase pathway. The single SGEF deletion in the father gives a unilateral subclinical foveal macular dysfunction despite the presence of the ABCA4 variant while the double deletion in the brother gives a more marked but bilateral subclinical phenotype in the brother who does not harbor the ABCA4 variant. This indicates a strong role of the SGEF homozygous deletion in the foveal macular defect as well as the CCA. Another clinical observation is the fact that on angiography the proband does not show the increased auto-fluorescence typical of ABCA4 Stargardt's disease due to A2E deposits giving rise to the lipofuscin deposits. This evidence goes against the ABCA4 defect as a major cause of the macular dystrophy.

Accordingly, albeit other gene loci may contribute to macular as well as corpus callosum defects, the role of SGEF is clearly established.

It has been shown, above, that SGEF has a key role in retinal macula, corpus callosum, hippocampus, liver and immune systems function and structure. Albeit the invention is not limited by the mechanism of action, it is of interest to note that these activities might involve SGEF's role as an activator of Rho GTPases. Some of these effects are mediated by the interaction with the actin cytoskeleton. Other effects might be initiated by receptor tyrosine kinases or G protein coupled receptors. SGEF is a key to modulation of Rho GTPase signaling which is a hub to promote normal neuronal connectivity and its regulation in response to extracellular signals and environment. While we have shown the key role of SGEF in promoting normal healthy inflammatory immune response and fever, its overexpression can be detrimental.

The scope of the invention also includes control of diseases caused by over-expression of SGEF or by excessive inflammation. The over-expressed SGEF might interact with the cytoskeleton to mediate cell movement, or the over-expressed SGEF might affect cell-cell interactions, transendothelial migration, cell division and multiplication, as well as cell transformation.

Because of its role in transendothelial migration SGEF can be understood as mediating a key step of the scavenging and prevention of atherosclerosis and plaques. Hagg, S. et al, PLoS Genet. 5(12):e1000754 (2009). (Epub 2009); Van Buul J D et. al., Arterioscler. Thromb. Vase. Biol. 24:824-833 (2004). Rho GTPases activity plays a key role in this process. Rolfe B E et al, 183(1): 1-16 (2005). Epub 2005 Jun. 27.) Thus, controlling/modulating SGEF, systemically, locally, or temporarily is a useful tool for the prevention and treatment of atherosclerosis.

Modulating SGEF must take into consideration its effects in multiple situations and the specific facts of a particular patient. For example, as noted above, SGEF has a role in activation of RHO GTPases and thus affect inflammatory cells like macrophage migration and phagocytosis, lipid uptake, a role in endothelial cells via PI3K intracellular signal transduction and also a role in vascular smooth cells in proliferation/migration and extracellular matrix uptake. It is the combination of these factors that contribute to endothelial dysfunction, coronary vasospasm, intimal hyperplasia and atherosclerosis.

However, the SGEF effect differs on the various systems. By way of example, both the inflammation response and the atherosclerosis mechanism involve active or overly active macrophages. As noted above, it was now observed that an SGEF deficient patient lacks an appropriate inflammatory response. This evidences a role for SGEF in normal macrophage function. Without limiting the invention to a particular mechanism of action, the absence of fever is due to a lack of trans-endothelial migration of macrophage or lack of macrophage activation of chemotaxix or phagocytosis. Accordingly, increased SGEF activity or expression is recommended for treatment of the patient lacking an adequate inflammatory response. A more controlled (reduced) macrophage activation would, on the other hand, lower the process of plaque formation in atherosclerosis. Accordingly, a somewhat diminished SGEF expression or activity level and a corresponding reduction in macrophage activation is beneficial to a patient prone to atherosclerosis, e.g. an obese patient, a patient with hypercholesterolemia or a diabetic.

Appropriate SGEF levels are critical for prevention and for control of multiple phenomena and a balance must be considered, under specific facts. For example, consider inflammation and atherosclerosis. Depending on the patient's profile, diagnosis and stage of disease development, one may choose to increase or decrease the level of SGEF activity. In severe cases of atherosclerosis, reduced SGEF activity levels are desirable. Generally an about 3% to about 80% reduction in the SGEF level is desirable, reduction by about 10% to about 50% yet more desirable, and a reduction by about 20% to about 50% more desirable yet. In a patient lacking a desirable inflammatory response, stimulation of SGEF is desired, in a controlled, perhaps temporary manner.

It has also recently been shown that low expression of SGEF is a marker of poor response to chemotherapy in ovarian cancer cells. Kim, S. W. et. al, OMICS 2011 Feb. 19 [Epub ahead of print].

Because of the pivotal role of SGEF in cell multiplication and growth, we conclude that the prolonged excess of SGEF gene expression or activation is a factor in carcinogenesis and cell transformation. Inhibitors of SGEF play a role in cancer treatments of prostate, ovarian and other cancers.

Another line of evidence is provided by inhibition of Rho kinase which is a downstream effector of GTPases as an effective tool to block cell migration Tsai C. C. et. al., Biochem. Pharmacol. 2011 Jan. 26. [Epub ahead of print]. This key property is another line of evidence that SGEF inhibitors are a useful treatment to block tumor cell migration.

Similarly gain of function mutations in the Ras GEF named SOS (“son of sevenless”) cause Noonan syndrome where cancer is a frequent complication. Roberts, A. E. et. al, Nature Genet. 39:70-74 (2007) and Tartaglia, M. et. al, Nature Genet. 39:75-79 (2007).

Rho kinase (ROCK1 and ROCK2) is a serine/threonine kinase that serves as a downstream effector of Rho GTPase. This class of kinases plays a key role in regulating the contractile tone of smooth muscle tissue through actin stress fibers via the phosphorylation of MLC (myosin light chain) in a calcium-independent manner. Myosin phosphorylation and the resultant increase in the contractile state are regulated through ROCK and subsequent vascular smooth muscle mediators such as Nitric oxide and endothelin. Rock inhibition leads to the relaxation of smooth muscle fibers. Experimental evidence indicates that inhibiting ROCK activity through topical and systemic ROCK inhibition could be beneficial for the treatment of increased intraocular pressure in patients with glaucoma, because both ROCK and Rho GTPase inhibitors can increase aqueous humor drainage via trabecular meshwork smooth muscle and ciliary muscle relaxation.

In ocular trabecular meshwork (TM) cells, the primary function of membrane-anchored Rho G-proteins is to promote filamentous actin stress fiber organization. Tian B. Exp Eye Res. 88:713-717 (2009). Activation of RhoG signaling enhances the contractile tone of TM cells, leading to slower rates of aqueous humor (AH) outflow and higher intraocular pressure (IOP). Rao V P and Epstein D L., Biodrugs 21:167-177 (2007). Inhibition of RhoG proteins or downstream RhoG effectors, such as Rho kinase, enhances AH outflow facility, thereby reducing IOP. Consequently, selective inhibitors of Rho signaling are aggressively being explored as potential therapeutic agents for the management of ocular hypertension. Von Zee C L. and Stubbs, E B. Jr. IO VS 52: 1676-1683 (2011); published ahead of print Jan. 6, 2011, doi: 10.1167/iovs. 10-6171.

In a parallel manner, another upstream activator of Rho G, the SGEF protein or protein expression inhibitor also is used as a therapeutic agent of increased intraocular pressure. In glaucoma the blockage of actin skeleton function will relax the trabecular meshwork and increase aqueous humor outflow thus decreasing the glaucoma severity. Since SGEF is a strong activator of RhoG we conclude that inhibition of SGEF in the anterior segment of the eye is useful as a smooth muscle and ciliary muscle relaxant and therefore a useful glaucoma or elevated intraocular treatment either topically or systemically.

An oral ROCK Inhibitor, Fasudil, is used in Japan for the prevention of cerebral vasospasm in patients with subarachnoid hemorrhage. Lau C. et. Ah, Br. J. Pharmacol. 2011 Feb. 10. doi: 10.1111/j.1476-5381.2011.01259. [Epub ahead of print]. Ditto, another upstream regulator of rho GTPase, the SGEF protein or protein expression inhibitor also is used as a therapeutic agent of increased intraocular pressure or glaucoma.

Fasudil along with other ROCK Inhibitors have been shown to reverse vasoconstriction, alter and improve blood flow after ischemic reperfusion injury, have neuroprotective properties, inhibit cellular proliferation, and inhibit inflammation. Preclinical models specific to cerebral and ocular injury are suggestive that ROCK Inhibitors could improve Retinal Ganglion Cell survival and axon regeneration, thus providing a potential benefit to patients with glaucomatous injury beyond IOP reduction. Local SGEF inhibition could have similar neuroprotective effects of retinal ganglion cells in glaucoma as well as ischemic reperfusion injury.

Rho kinase inhibition decreases liver fibrosis and SGEF inhibition has a similar effect of preventing liver fibrosis if delivered directly to the liver, for example as a conjugate to Glucose 6 phosphate human serum albumin which is selectively taken up by stellate liver cells. Van Beuge M. et. al., J. Pharmacol. Exp. Ther. (2011). [Epub ahead of print].) A SGEF inhibitor will thus have a protective effect against liver fibrosis.

Rho kinase inhibition has also been associated with treatment of osteoarthritis in animal models. Takeshita N., J. Pharmacol. Sci. 2011 Feb. 16 [Epub ahead of print] and as a preventative and curative treatment of osteoarthritis and joint inflammatory processes, locally and systemically.

### ADDITIONAL REFERENCES

Saraste, M. and Hyvonen, M. “Pleckstrin homology domains: a fact file” Curr. Opin. Struct. Biol. 5(3):403-408 (1995).
Riddihough, G. “More meanders and sandwiches” Nat. Struct. Biol. 1(11):755-757 (1994).
Hart M J, Eva A, Evans T, Aaronson S A, Cerione R A Catalysis of guanine nucleotide exchange on the CDC42Hs protein by the dbl oncogene product; Nature. 354:311-314(1991)
Tan E C, et. al, The human active breakpoint cluster region-related gene encodes a brain protein with homology to guanine nucleotide exchange proteins and GTPase-activating proteins; J. Biol. Chem. 268:27291-27298 (1993).
Soisson S M, Nimnual A S, Uy M, Bar-Sagi D, Kuriyan J. Crystal structure of the Dbl and pleckstrin homology domains from the human Son of sevenless protein. Cell; 95:259-268(1998).
Ridley, A. J. “Rho GTPases and actin dynamics in membrane protrusions and vesicle trafficking”. Trends Cell Biol. 16(10):522-9(2006).
RHOG ras homolog gene family, member G (rho G) [Homo sapiens] Gene ID: 391, updated on 6 Mar. 2011; www.ncbi.nlm.nih.gov/sites/entrez?db=gene&Cmd=ShowDetailView&TermToSearch=391, last viewed on Mar. 6, 2011.
Vincent S. et al. Growth-regulated expression ofrhoG, a new member of the ras homolog gene family. Mol. Cell Biol. 12(7):3138\hat{ }48 (1992).
Condliffe A M et al “RhoG regulates the neutrophil NADPH oxidase”, J. Immunol. 176(9): 5314-20 (2006).
Vigorito E. et al. “RhoG regulates gene expression and the actin cytoskeleton in lymphocytes”. Oncogene 22(3): 330\hat{ }2 (2003).
Katoh H. et al., Small GTPase RhoG is a key regulator for neurite outgrowth in PC12 cells Mol. Cell Biol. 20(19): 7378-87 (2000).
Van Buul J D, et al “RhoG regulates endothelial apical cup assembly downstream from ICAM1 engagement and is involved in leukocyte trans-endothelial migration” J. Cell Biol. 178(7): 1279-93 (2007).
Katoh. et al. “Activation of Racl by RhoG regulates cell migration” J. Cell. Sci. 119(1): 56-65 (2006).
Boureux A. et al. “Evolution of the Rho family of ras-like GTPases in eukaryotes”. Mol. Biol. Evol. 24(1): 203-16 (2007).
Murga C. et al., Racl and RhoG promote cell survival by the activation of PI3K and Akt, independently of their ability to stimulate JNK and NF-κB, Oncogene 21(2): 207-16 (2002).
Prieto-Sanchez R M, et al. “Involvement of the Rho/Rac family member RhoG in caveolar endocytosis”. Oncogene 25(21):2961-73 (2006).
Yamaki N, et al., RhoG regulates anoikis through a phosphatidylinositol 3-kinase-dependent mechanism, Exp. Cell Res. 313(13): 2821-32 (2007).
Blangy A, et al. “TrioGEFl controls Rac- and Cdc42-dependent cell structures through the direct activation of RhoG”. J. Cell Sci. 113(Pt 4): 729-39 (2000).
Bellanger J, et al. “The two guanine nucleotide exchange factor domains of Trio link the Racl and the RhoA pathways in vivo”. Oncogene 16(2): 147-52(1998).
Estrach S. et al. “The Human Rho-GEF trio and its downstream target GTPase RhoG are involved in the NGF pathway, leading to neurite outgrowth”. Curr. Biol. 12(4): 307-12 (2002).
deBakker CD. et al. “Phagocytosis of apoptotic cells is regulated by a UNC-73/TRIO-MIG-2/RhoG signaling module and armadillo repeats of CED-12/ELMO”. Curr. Biol. 14(24): 2208-16 (2004)
Schuebel K E, et al. “Phosphorylation-dependent and constitutive activation of Rho proteins by wild-type and oncogenic Vav-2”. EMBO J. 17(22): 6608-21 (1998).
Movilla N and Bustelo X R. Biological and regulatory properties of Vav-3, a new member of the Vav family of oncoproteins, Mol. Cell. Biol. 19(11): 7870-85(1999).
Zalcman G. et, al., RhoGDI-3 is a new GDP dissociation inhibitor (GDI).
Identification of a non-cytosolic GDI protein interacting with the small GTP-binding proteins RhoB and RhoG, J. Biol. Chem. 271(48): 30366-74 (1996).
Cote J, Vuori K. “GEF what? Dockl80 and related proteins help Rac to polarize cells in new ways”. Trends Cell Biol. 17(8): 383-93(2007).
Brugnera E, et al. “Unconventional Rac-GEF activity is mediated through the Dockl80-ELMO complex”. Nat. Cell Biol. 4 (8): 574-82(2002).
Lu M, et al. “PH domain of ELMO functions in trans to regulate Rac activation via Dock!80”. Nat. Struct. Mol. Biol. 11(8): 756-62(2004).
Katoh, H. and Negishi, M. “RhoG activates Racl by direct interaction with the Dockl80-binding protein Elmo”. Nature 424(6947): 461-64(2003).
Vignal E, Blangy A, Martin M, et al., Kinectin is a key effector of RhoG microtubule-dependent cellular activity, Mol. Cell Biol. 21(23): 8022-34 (2001).
Taviaux S A, Vincent S, Fort P, Demaille J G. “Localization of ARHG, a member of the RAS homolog gene family, to I lpl5.5-l lpl5.4 by fluorescence in situ hybridization.” Genomics 16(3): 788-90 (1993).
Prieto-Sanchez R M, Bustelo X R. “Structural Basis for the Signaling Specificity of RhoG and Racl GTPases.”. J. Biol. Chem. 278(39): 37916-25(2003).
Patel J C, Galan J E. “Investigating the Function of Rho Family GTPases during Salmonella/Host Cell interactions.”. Meth. Enzym. 439: 145-58(2008).
Meller N, et al. “CZH proteins: a new family of Rho-GEF s.”. J. Cell. Sci. 118(Pt 21): 4937-46(2005).
Lu M, et al. “A Steric-Inhibition Model for Regulation of Nucleotide Exchange via the Dockl80 Family of GEF s.”. Curr. Biol. 15(4): 371-77(2005).
Jankowski A, et al. “Capture of an activated complex from the surface of live cells by affinity receptor chromatography.” Anal. Biochem. 380:235 (2008).
Vigorito E, et al. “Immunological Function in Mice Lacking the Rac-Related GTPase RhoG.”. Mol. Cell. Biol. 24(2): 719-29(2004).
Meller J, et al. “Endogenous RhoG is dispensible for integrin-mediated cell spreading but contributes to Rac-independent migration.” J. Cell. Sci. 121(Pt 12): 1981-89(2008).
Miki T, et al. “Oncogene ect2 is related to regulators of small GTP-binding proteins.” Nature 362(6419): 462-5 (1993).
Le Gallic L, and Fort P. “Structure of the human ARHG locus encoding the Rho/Rac-like RhoG GTPase.” Genomics 42(1): 157-60(1997).
Booden M A, Campbell S L, Der C J. “Critical but distinct roles for the pleckstrin homology and cysteine-rich domains as positive modulators of Vav2 signaling and transformation.” Mol. Cell. Biol. 22(8): 2487-97 (2002).
Skowronek K R, Guo F, Zheng Y, Nassar N. “The C-terminal basic tail of RhoG assists the guanine nucleotide exchange factor trio in binding to phospholipids.” J. Biol. Chem. 279(36): 37895-907(2004).
Hiramoto K, Negishi M, Katoh H. “Dock4 is regulated by RhoG and promotes Rac-dependent cell migration.” Exp. Cell Res. 312(20): 4205-16 (2007).
Gumienny T L, Brugnera E, Tosello-Trampont A et al, “CED-12/ELMO, a Novel Member of the Crkll/Dockl 80/Rac Pathway, Is Required for Phagocytosis and Cell Migration”. Cell 107(1): 27-41(2001).
Kunisaki Y, et al, “DOCK2 is a Rae activator that regulates motility and polarity during neutrophil chemotaxis” J. Cell. Biol. 174(5): 647-52 (2006).
Lu M, Ravichandran K S. “Dockl 80-ELMO Cooperation in Rae Activation.” Meth. Enzym. 406:388-402 (2006).

Multiple aspects of the invention were illustrated by proposing particular mechanisms of actions which appear preferred mechanisms. However, the invention's scope is not limited by a mechanism of action.

All references, including publications, patent applications, patents, and website content cited herein are hereby incorporated by reference to the same extent as if each reference were individually and specifically indicated to be incorporated by reference and was set forth in its entirety herein.

The use of the terms “a” and “an” and “the” and similar referents in the context of describing the invention are to be construed to cover both the singular and the plural, unless otherwise indicated herein or clearly contradicted by context.

Unless otherwise indicated herein, recitation of ranges of values herein are merely intended to serve as a shorthand method of referring individually to each separate value falling within the range, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.,” or “such as”) provided herein, is intended merely to better illuminate the invention and does not pose a limitation on the scope of the invention, unless otherwise claimed. No language in the specification should be construed as indicating any non-claimed element as essential to the practice of the invention.

1.-28. (canceled)

29. A method of treating an individual having a defect in an SGEF gene located at human chromosome 3q25.2 (3q25.2) producing RhoG phosphorylation more than two times the activity of a natural SGEF protein, and manifesting an inflammatory disease state, comprising administering at least one Src Homology 3-containing Guanine Nucleotide Exchange Factor (SGEF) modulator to decrease SGEF activity by between about 3% to about 80%, and a pharmaceutical carrier to
wherein the SGEF modulator is selected from the group consisting of:
(i) an antibody targeting a full length wild-type SGEF protein encoded by the SGEF gene located at 3q25.2, wherein the antibody is further structurally defined as a humanized antibody, a single chain antibody, or an ab2s fragment.
(ii) an siRNA modulator targeting an mRNA translating into a full length wild-type SGEF protein corresponding to the protein encoded by the SGEF gene located at 3925.2, and
(iii) an antisense RNA modulator targeting an mRNA translating into a full length wild-type SGEF protein corresponding to the protein encoded by the SGEF gene located at 3925.2.

30. The method of claim 29, wherein the inflammatory disease state is selected from the group consisting of glaucoma, and an atherosclerosis condition with obesity and/or hypercholesterolemia.

31. The method of claim 30, wherein the inflammatory disease state is an atherosclerosis condition with obesity and/or hypercholesterolemia, and the modulator to decrease SGEF activity is an antibody targeting a full length wild-type SGEF protein encoded by the SGEF gene located at 3925.2, wherein the antibody is further structurally defined as a humanized antibody, a single chain antibody, or an ab2s fragment.

32. The method of claim 31, where the modulator reduces the SGEF activity reduced by between about 10% to about 50%.

33. The method of claim 30, wherein the defect in an SGEF gene located at 3q25.2 is a heterozygous defect decreasing expression of the SGEF gene located at 3q25.2 or homozygous defect abolishing expression of the SGEF gene located at 3q25.2.

34. The method of claim 34, wherein and the modulator is an antibody targeting a full length wild-type SGEF protein encoded by the SGEF gene located at 3q25.2.

35. A method of treating an individual having a defect in an SGEF gene located at human chromosome 3q25.2 (3q25.2) producing RhoG phosphorylation less than 70% of the activity of a natural SGEF protein, and manifesting a disease state selected from the group consisting of a retinal macular anomaly and a corpus callosum deficiency, comprising administering at least one Src Homology 3-containing Guanine Nucleotide Exchange Factor (SGEF) modulator to increase SGEF activity and a pharmaceutical carrier; and the SGEF modulator is selected from the group consisting of
(i) a full length, wild-type SGEF protein corresponding to the protein encoded by the SGEF gene located at 3925.2,
(ii) a gene encoding the full length wild-type SGEF protein corresponding to the protein encoded by the SGEF gene located at 3q25.2, and
(iii) an SGEF protein comprising a DBL homology (DH) domain followed by Plekstrin homology (PH) domain, a N-terminal proline rich domain, a vacuolar domain, and a Src homology (SRC) domain located at the C-terminal.

36. The method of claim 35, wherein the defect in the SGEF gene located at 3q25.2 produces phosphorylation of less than 50% of the activity of a natural SGEF protein.

37. The method of claim 35, where the individual manifests a retinal macular anomaly.

38. The method of claim 37, wherein the modulator is a full length, wild-type SGEF protein corresponding to the protein encoded by the SGEF gene located at 3925.2 or a gene encoding the full length wild-type SGEF protein corresponding to the protein encoded by the SGEF gene located at 3925.2.

39. The method of claim 37, where the modulator is a an SGEF protein comprising a DBL homology (DH) domain followed by Plekstrin homology (PH) domain, a N-terminal proline rich domain, a vacuolar domain, and a Src homology (SRC) domain located at the C-terminal.


SEQUENCE LISTING (5 sequences)

SEQ ID NO: 1 - protein, 751 aa, Homo sapiens
MDGESEVDFSSNSITPLWRRRSIPQPHQVLGRSKPRPQSYQSPNGLLITDFPVEDGGTLLAAQIPAQVPTASDSRTVHRSPLLLGAQRRAVANGGTASPEYRAASPRLRRPKSPKLPKAVPGGSPKSPANGAVTLPAPPPPPVLRPPRTPNAPAPCTPEEDLTGLTASPVPSPTANGLAANNDSPGSGSQSGRKAKDPERGLFPGPQKSSSEQKLPLQRLPSQENELLENPSVVLSTNSPAALKVGKQQIIPKSLASEIKISKSNNQNVEPHKRLLKVRSMVEGLGGPLGHAGEESEVDNDVDSPGSLRRGLRSTSYRRAVVSGFDFDSPTSSKKKNRMSQPVLKVVMEDKEKFSSLGRIKKKMLKGQGTFDGEENAVLYQNYKEKALDIDSDEESEPKEQKSDEKIVIHHKPLRSTWSQLSAVKRKGLSQTVSQEERKRQEAIFEVISSEHSYLLSLEILIRMFKNSKELSDTMTKTERHHLFSNITDVCEASKKFFIELEARHQNNIFIDDISDIVEKHTASTFDPYVKYCTNEVYQQRTLQKLLATNPSFKEVLSRIESHEDCRNLPMISFLILPMQRVTRLPLLMDTICQKTPKDSPKYEVCKRALKEVSKLVRLCNEGARKMERTEMMYTINSQLEFKIKPFPLVSSSRWLVKRGELTAYVEDTVLFSRRTSKQQVYFFLFNDVLIITKKKSEESYNVNDYSLRDQLLVESCDNEELNSSPGKNSSTMLYSRQSSASQSPLYSDSP

SEQ ID NO: 2 - protein, 446 aa, Homo sapiens
MDGESEVDFSSNSITPLWRRRSIPQPHQVLGRSKPRPQSYQSPNGLLITDFPVEDGGTLLAAQIPAQVPTASDSRTVHRSPLLLGAQRRAVANGGTASPEYRAASPRLRRPKSPKLPKAVPGGSPKSPANGAVTLPAPPPPPVLRPPRTPNAPAPCTPEEDLTGLTASPVPSPTANGLAANNDSPGSGSQSGRKAKDPERGLFPGPQKSSSEQKLPLQRLPSQENELLENPSVVLSTNSPAALKVGKQQIIPKSLASEIKISKSNNQNVEPHKRLLKVRSMVEGLGGPLGHAGEESEVDNDVDSPGSLRRGLRSTSYRRAVVSGFDFDSPTSSKKKNRMSQPVLKVVMEDKEKFSSLGRIKKKMLKGQGTFDGEENAVLYQNYKEKALDIDSDEESEPKEQKSDEKIVIHHKPLRSTWSQLSAVKRKVILIVGFMEMKDGRLRGGK

SEQ ID NO: 3 - protein, 110 aa, Homo sapiens
MDGESEVDFSSNSITPLWRRRSIPQPHQVLGRSKPRPQSYQSPNGLLITDFPVEDGGTLLAAQIPAQVPTASDSRTVHRSPLLLGAQRRAVANGGTASPEYRAASPRLRR

SEQ ID NO: 4 - protein, 154 aa, Homo sapiens
MKSLILLQGRTAPQCSIQDRALPVSHLFTLTVLSNHANEKVEMLLGAETQSERARWITALGHSSGKPPADRTSLTQVEIVRSFTAKQPDELSLQVADVVLIYQRVSDGWYEGERLRDGERGWFPMECAKEITCQATIDKNVERMGRLLGLETNV

SEQ ID NO: 5 - protein, 137 aa, Homo sapiens [sequence omitted]